(19) United States
(12) Patent Application Publication
(10) Pub. No.: US 2026/0083770 A1
(43) Pub. Date: Mar. 26, 2026

(54) RNA COMPLEXES AND NANOSTRUCTURES FOR TREATMENT OF CANCER METASTASIS

(71) Applicant: OHIO STATE INNOVATION FOUNDATION, Columbus, OH (US)

(72) Inventors: Peixuan GUO, Columbus, OH (US); Xin LI, Columbus, OH (US); Daniel BINZEL, Columbus, OH (US); Kai JIN, Columbus, OH (US)

(21) Appl. No.: 19/110,315

(22) PCT Filed: Sep. 28, 2023
(86) PCT No.: PCT/US2023/075448

§ 371 Date: Mar. 10, 2025

Related U.S. Application Data
(60) Provisional application No. 63/377,390, filed on Sep. 28, 2022.
(60) Provisional application No. 63/511,969, filed on Jul. 5, 2023.

(51) Int. Cl. A61K 31/7105; A61K 31/7115; A61K 31/713; A61K 47/54; A61K 47/69; A61P 35/04; C12N 15/113

(52) U.S. Cl. CPC A61K 31/7105; A61K 31/7115; A61K 31/713; A61K 47/549; A61K 47/6929; A61P 35/04; C12N 15/1135; C12N 2310/11; C12N 2310/335

(57) ABSTRACT

Disclosed herein are compositions and methods for one step CMC production of RNA therapeutic complexes (nanostructures) that contain nucleoside analogues. In some embodiments, the nucleoside analogues are incorporated into RNA oligonucleotides that self-assemble into an RNA complex during RNA synthesis in a one-step production. Therefore, no additional conjugation or synthesis processes are required.

### CROSS-REFERENCE TO RELATED APPLICATIONS

This application claims benefit of U.S. Provisional Application No. 63/377,390, filed Sep. 28, 2022, and of U.S. Provisional Application No. 63/511,969, filed Jul. 5, 2023, which is hereby incorporated herein by reference in its entirety.

### STATEMENT REGARDING FEDERALLY SPONSORED RESEARCH OR DEVELOPMENT

This invention was made with Government Support under Grant Nos. CA207946 and EB019036 awarded by the National Institutes of Health. The Government has certain rights in the invention.

### BACKGROUND

The metastasis has been a major outcome leading to patient death. siRNA has been a long-term dream for the treatment of cancer. However, currently, there is not a single siRNA drug has been approved by FDA for the treatment of cancer. The major hurdle for the ineffectiveness of the treatment is the lack of effective procedures for the delivery of the siRNA to cancer cells in vivo and the trapping of siRNA in endosomes.

### SUMMARY

Disclosed herein is a special design for effective delivery of inhibitory RNA molecules (e.g. siRNA) to cells in vivo and a novel method for the release of the siRNA in the RNA nano-complex via a special design for cleaving the escorting RNA sequence. In particular, disclosed herein is a therapeutic RNA complex that involves a plurality of synthetic double-stranded RNA oligonucleotides, each synthetic double-stranded RNA oligonucleotide comprising a sense strand having a 5′ end and a 3′ end, an antisense strand having a 5′ end and a 3′ end, and a cholesterol molecule conjugated to the 3′ end of the sense strand, wherein the plurality of synthetic double-stranded RNA oligonucleotides are in an aggregate with the cholesterol molecules at the center. The sense strand comprises more than 25 nucleotides with the 2′-positions of all the pyrimidines at the sense strand do not contain oxygen. The antisense stand comprises an siRNA sequence with 18-25 normal nucleotides. wherein the sense strand is longer than the antisense strand. At least 95% of the nucleotides in the antisense strand are hybridized to complementary nucleotides of the sense strand.

In some embodiments, the nucleoside analogs comprise 2′-difluoro-deoxypyrimidine, floxuridine (5-fluorodeoxyuridine, UB5F) nucleotides, gemcitabine (2′, 2′-difluoro-2′-deoxycytidine, dFdC, CR2FF) nucleotides, or a combination thereof. In some embodiments, the 5′ end of the sense strand are modified with GalNAc (N-acetylgalactosamine), UAMC-1110 (SP-13786; (S)—N-(2-(2-Cyano-4,4-difluoropyrrolidin-1-yl)-2-oxoethyl)quinoline-4-carboxamide), FA (Folate), or DCL (N—[N— [(S)-1,3-dicarboxypropyl]carbamoyl]-(S)-lysine).

Also described herein are RNA nanostructures that can be composed of one or more synthetic RNA oligonucleotides that are designed (or configured) to self-assemble into the RNA nanostructures. When assembled, the RNA nanostructures can be composed of double-stranded arms (DAs) that can be arranged around a core domain. In addition, at least one of the RNA oligonucleotides in the RNA nanostructure contains at least 1, 2, 3, 4, 5, 6, 7, 8, 9, or 10 floxuridine (5-fluorodeoxyuridine, UB5F) nucleotides, gemcitabine (2′, 2′-difluoro-2′-deoxycytidine, dFdC, CR2FF) nucleotides, or a combination thereof. In some embodiments, at least 1, 2, 3, or 4 of the synthetic RNA oligonucleotides have an amino acid sequence that is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS:1-448. In some embodiments, synthetic RNA oligonucleotides containing nucleoside analogues are combined with un-modified RNA oligonucleotides to form the RNA nanostructure. For example, the at least 1, 2, 3, or 4 synthetic RNA oligonucleotides that have an amino acid sequence that is about 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS:1-448 can be combined with at least 1, 2, 3, 4, 5, or 6 synthetic RNA oligonucleotides that have an amino acid sequence that is about 80-100% identical to any one of SEQ ID NOS:298-394. Suitable combinations of these oligonucleotides to form RNA nanoparticles are described herein.

Also disclosed herein are compositions and methods for one step CMC production of RNA therapeutic complexes (nanostructures) that contain nucleoside analogues. In some embodiments, the nucleoside analogues are incorporated into RNA oligonucleotides that self-assemble into an RNA complex during RNA synthesis in a one-step production. Therefore, no additional conjugation or synthesis processes are required. The disclosed compositions and methods enable the large industry-scale production of pure RNA therapeutics without the complexity of drug conjugation. In some embodiments, the RNA complexes contains at least two kinds of nucleoside analogues. In some embodiments, the RNA complexes contains at least 20, 21, 22, 23, 24, 25, 26, 27, 28, 29, 30, 31, 32, 33, 34, 35, 36, 37, 38, 39, 40 nucleoside analogues distributed within the RNA complex. In some embodiments, the RNA complexes does not contain uridine or cytidine nucleotides.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates modified nucleotides used in the disclosed RNA nanoparticles.

FIG. 2 shows results of a cytotoxicity study of RNA-UB5F nanoparticles in breast cancer cell line.

FIG. 3 shows results of a cytotoxicity study of RNA-CR2FF nanoparticles in breast cancer cell line.

FIG. 4 shows results of a cytotoxicity study of RNA-CR2FF-UB5F nanoparticles in breast cancer cell line.

FIG. 5 shows Survivin-Double strands structure: Sense-Strand with 3′ cholesterol and gemcitabine (as Y)

FIG. 6 shows Survivin-Double strands structure: Sense-Strand with 3′ cholesterol and gemcitabine (as Y) and Anti sense-Strand with extension linked to it

FIG. 7 shows Survivin-Double strands structure: Sense-Strand with 3′ cholesterol and gemcitabine (as Y) and Anti sense-Strand has an adjacent extension RNA structure

FIG. 8 shows RRM2-Double strands structure: Sense-Strand with 3′ cholesterol and gemcitabine (as Y)

FIG. 9 shows RRM2-Double strands structure: Sense-Strand with 3′ cholesterol and gemcitabine (as Y) and Anti sense-Strand with extension linked to it

FIG. 10 shows RRM2-Double strands structure: Sense-Strand with 3′ cholesterol and gemcitabine (as Y) and Anti sense-Strand has an adjacent extension RNA structure

FIG. 11 Curves showing results of a weight change study of RNA-CR2FF-Cholesterol nanoparticles in human colorectal adenocarcinoma cell lung metastasis animal model.

FIG. 12 Histogram showing results of a weight change study of RNA-CR2FF-Cholesterol nanoparticles in human colorectal adenocarcinoma cell lung metastasis animal model.

FIG. 13 shows results of in vivo metastasis inhibition study of RNA-CR2FF-Cholesterol nanoparticles in human colorectal adenocarcinoma cell lung metastasis animal model.

FIG. 14 shows results of ex vivo metastasis inhibition study of RNA-CR2FF-Cholesterol nanoparticles in human colorectal adenocarcinoma cell lung metastasis animal model.

FIG. 15 shows design of RNA/Drug/siRNA complexes to form into RNA micelles. RNA-micelle carrying siRNA (survivin or RRM2 siRNA) and multiple copies of gemcitabine.

FIGS. 16A and 16B show lung tropic training of HT29 cells resulted into two major changes. FIGS. 16A and 16B show speed of metastatic tumor progression (from 5 month to 1 month after injection (FIG. 16A) and increase in metastatic burden (FIG. 16B) as imaged by bioluminescence scans.

FIGS. 17A to 17D show in vivo delivery of RNA micelles for treatment of CRC lung metastasis. FIG. 17A shows whole body bioluminescence imaging of mice following RNA micelle delivery demonstrating decreased tumor GFP signaling. FIG. 17B shows tumor bioluminescence imaging following RNA micelle delivery demonstrating GFP decrease of tumor sites. FIG. 17C shows Ki-67 staining demonstrating proliferation of excised tumors following treatment. FIG. 17D shows caspase 3 staining demonstrating apoptosis signaling of excised tumors following treatment.

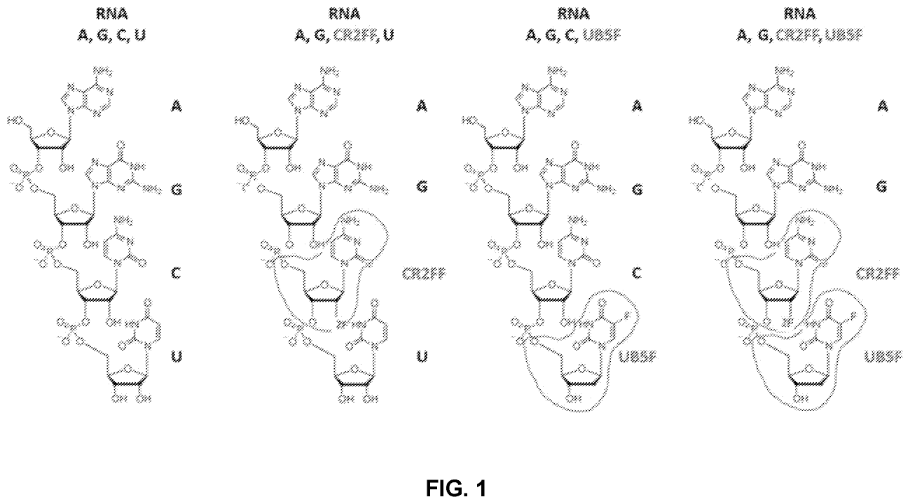

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

As used herein, “self-assembly” refers to the ability of nucleic acids (and, in some instances, preformed nucleic acid nanostructures (e.g., crystals)) to anneal to each other, in a sequence-specific manner, in a predicted manner and without external control. In some aspects, nucleic acid nanostructure self-assembly methods include combining nucleic acids (e.g., single-stranded nucleic acids, or oligonucleotides) in a single vessel and allowing the nucleic acids to anneal to each other, based on sequence complementarity. In some aspects, this annealing process involves placing the nucleic acids at an elevated temperature and then reducing the temperature gradually in order to favor sequence-specific binding. Various nucleic acid nanostructures or self-assembly methods are known and described herein.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,
where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “siRNA” as used herein refers to a short inhibitory polynucleotide that can post-transcriptionally silence gene expression in a cell. siRNA are normally double stranded, but only one strand of the siRNA is involved in gene silencing.

### Inhibitory RNA

Disclosed herein are compositions and methods for stabilizing and delivering inhibitory nucleic acids. Inhibitory nucleic acids or “siNA”, as used herein, is defined as a short interfering nucleic acid. Examples of siNA include but are not limited to RNAi, double-stranded RNA, and siRNA. A siNA can inhibit the transcription or translation of a gene in a cell. A siNA may be from 16 to 1000 or more nucleotides long, and in certain embodiments from 18 to 100 nucleotides long. In certain embodiments, the siNA may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. The siNA may comprise a nucleic acid and/or a nucleic acid analog. Typically, a siNA will inhibit the translation of a single gene within a cell; however, in certain embodiments, a siNA will inhibit the translation of more than one gene within a cell.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90 to 100, 150, 200, 250, 300, 350, 400, 450, 500 or more contiguous nucleobases, including all ranges therebetween. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siNA (e.g., siRNA) are well known in the art. For example, siRNA and double-stranded RNA have been described in U.S. Pat. Nos. 6,506,559 and 6,573,099, as well as in U.S. Patent Applications 2003/6051263, 2003/0055020, 2004/0265839, 2002/0168707, 2003/0159161, and 2004/0064842, all of which are herein incorporated by reference in their entirety.

In some cases, siRNA are capable of (i) binding to the respective mRNA, (ii) interfere with signaling and/or (iii) inhibit proliferation cancer or tumor cell.

Typically, introduction of double-stranded RNA (dsRNA), which may alternatively be referred to herein as small interfering RNA (siRNA), induces potent and specific gene silencing, a phenomena called RNA interference or RNAi. This phenomenon has been extensively documented in the nematode C. elegans (Fire et al., 1998), but is widespread in other organisms, ranging from trypanosomes to mouse. Depending on the organism being discussed, RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80, 85, 90, 95, 98,% or even 100% identity between the sequence of the siRNA and the gene to be inhibited. Sequences less than about 80% identical to the target gene are substantially less effective. Thus, the greater homology between the siRNA of FAK, EphA2, and/or β2AR and the FAK, EphA2, and/or β2AR gene whose expression is to be inhibited, the less likely expression of unrelated genes will be affected.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 20040019001 and U.S. Pat. No. 6,673,611 (each of which is incorporated by reference in its entirety). Collectively, all such altered nucleic acids or RNAs described above are referred to as modified siRNAs.

Introduction of siRNA into cells can be achieved by methods known in the art, including for example, microinjection, electroporation, or transfection of a vector comprising a nucleic acid from which the siRNA can be transcribed. Alternatively, a siRNA can be directly introduced into a cell in a form that is capable of binding to target mRNA transcripts. To increase durability and membrane-permeability the siRNA may be combined or modified with liposomes, poly-L-lysine, lipids, cholesterol, lipofectine or derivatives thereof. In certain aspects cholesterol-conjugated siRNA can be used (see, Song et al., 2003).

### Therapeutic RNA Complex

Disclosed herein is a therapeutic RNA complex for delivery of an siRNA to a cell in a subject. The therapeutic RNA complex involves a plurality of synthetic double-stranded RNA oligonucleotides, each synthetic double-stranded RNA oligonucleotide comprising a sense strand having a 5′ end and a 3′ end, an antisense siRNA strand having a 5′ end and a 3′ end, and a cholesterol molecule conjugated to the 3′ end of the sense strand. In some embodiments, the sense strand comprises 3, 4, 5, 6, 7, 8, 9, 10, 11, 12, 13, 14, 15, 16, 17, 18, 19, 20, or more nucleoside analogs, such as floxuridine (5-fluorodeoxyuridine, UB5F) nucleotides, gemcitabine (2′, 2′-difluoro-2′-deoxycytidine, dFdC, CR2FF) nucleotides, or a combination thereof. These nucleoside analogs protect the sense strand from RNase degradation but are also targets for digestion once inside the cell.

In some embodiments, the antisense strand is an anticancer siRNA affecting survival or apoptosis. For example, the antisense strand can be a survivin siRNA or RRM2 siRNA. In some embodiments the survivin siRNA has the nucleic acid sequence SEQ ID NO:1. In some embodiments the RRM2 siRNA has the nucleic acid sequence SEQ ID NO:2.

In some embodiments, the sense strand is the same length or longer than the antisense strand. Importantly, at least 85%, 90%, 95%, or 100% of the nucleotides in the antisense strand should be hybridized to complementary nucleotides of the sense strand to protect the siRNA from RNase digestion. Once inside the cell, the sense strand is digested, thereby releasing the siRNA in the cell.

In some embodiments, the plurality of synthetic double-stranded RNA oligonucleotides are in an aggregate with the cholesterol molecules at the center. This is referred to herein as “RNA micelles.”

In some embodiments, the RNA complex further includes GalNAc (N-acetylgalactosamine), UAMC-1110 (SP-13786; (S)—N-(2-(2-Cyano-4,4-difluoropyrrolidin-1-yl)-2-oxoethyl)quinoline-4-carboxamide), FA (Folate), or DCL (N—[N—[(S)-1,3-dicarboxypropyl]carbamoyl]-(S)-lysine) on the 5′ end of the sense strand.

Therefore, disclosed herein is a synthetic double-stranded RNA oligonucleotide involving a sense strand having a 5′ end and a 3′ end and an antisense siRNA strand having a 5′ end and a 3′ end, wherein the sense strand comprises 3, 4, 5, 6, 7, 8, 9, 10, 11, 12, 13, 14, 15, 16, 17, 18, 19, 20, or more floxuridine (5-fluorodeoxyuridine, UB5F) nucleotides, gemcitabine (2′, 2′-difluoro-2′-deoxycytidine, dFdC, CR2FF) nucleotides, or a combination thereof.

Also disclosed herein is a method for stabilizing an siRNA for therapeutic delivery, the method involving producing a plurality of synthetic single stranded RNA oligonucleotides complementary to the siRNA sequence, wherein each of the the RNA oligonucleotides comprises 3, 4, 5, 6, 7, 8, 9, 10, 11, 12, 13, 14, 15, 16, 17, 18, 19, 20, or more nucleoside analogs configured to prevent RNase degradation, and wherein each of the RNA oligonucleotides comprises a cholesterol moiety conjugated to its 3′ end. The method can then involve contacting the siRNA with the RNA oligonucleotides under conditions suitable to allow hybridization of the siRNA to the RNA oligonucleotides to produce a double-stranded RNA molecules that aggregate together with the cholesterol molecules at the center.

### RNA Nanostructures

Also described herein are RNA nanostructures that can be composed of one or more synthetic RNA oligonucleotides that are designed (or configured) to self-assemble into the RNA nanostructures. When assembled, the RNA nanostructures can be composed of double-stranded arms (DAs) that can be arranged around a core domain.

In some embodiments, at least one of the RNA oligonucleotides in the RNA nanoparticle contains at least 1, 2, 3, 4, 5, 6, 7, 8, 9, or 10 floxuridine (5-fluorodeoxyuridine, UB5F) nucleotides, gemcitabine (2′, 2′-difluoro-2′-deoxycytidine, dFdC, CR2FF) nucleotides, or a combination thereof.

In some embodiments, the RNA oligonucleotides are modified at the 5′ end with GalNAc (N-acetylgalactosamine), UAMC-1110 (SP-13786; (S)—N-(2-(2-Cyano-4,4-difluoropyrrolidin-1-yl)-2-oxoethyl)quinoline-4-carboxamide), FA (Folate), or DCL (N—[N—[(S)-1,3-dicarboxypropyl]carbamoyl]-(S)-lysine).

In some embodiments, the disclosed RNA oligonucleotide has one of the following sequences:

3WJ-A with floxuridine and gemcitabine and
derivatives:
5′-XXGYYAXGXGXAXGXGGG-3′;
(targeting aptamer sequences in lower case);
(SEQ ID NO: 6)
5′-XXGYYAXGXGXAXGXGGGGgccuuaguaacgugcuuugaugucgauu
cgacaggaggc-3′
(SEQ ID NO: 7)
5′-XXGYYAXGXGXAXGXGGGGGGACCGAAAAAGACCUGACUUCUAUACU
AAGUCUACGUUUCCC-3′;
(targeting aptamer sequences in lower case);
(SEQ ID NO: 8)
5′-XXGYYXGXGXAXGXGGGgcgacugguuacccggucg-3′
(targeting aptamer sequences in lower case);
(SEQ ID NO: 9)
5′-XXGYYAXGXGXAXGXGGGcagaacguauacuauucug-3′
5′-GalNAc-XXGYYAXGXGXAXGXGGG-3′;
5′-FA-XXGYYAXGXGXAXGXGGG-3′;
5′-DCL-XXGYYAXGXGXAXGXGGG-3′;
5′-UAMC1110-XXGYYAXGXGXAXGXGGG-3′;
3WJ-B with floxuridine and gemcitabine and
derivatives:
5′-YYYAYAXAYXXXGXXGAXYY-3′;
(targeting aptamer sequences in lower case);
(SEQ ID NO: 10)
5′-YYYAYAXAYXXXGXXGAXYYgccuuaguaacgugcuuugaugucgau
ucgacaggaggc-3′
(SEQ ID NO: 11)
5′-YYYAYAXAYXXXGXXGAXYYGGGACCGAAAAAGACCUGACUUCUAUA
CUAAGUCUACGUUUCCC-3′;
(targeting aptamer sequences in lower case);
(SEQ ID NO: 12)
5′-YYYAYAXAYXXXGXXGAXYYgcgacugguuacccggucg-3′
(targeting aptamer sequences in lower case);
(SEQ ID NO: 13)
5′-YYYAYAXAYXXXGXXGAXYYcagaacguauacuauucug-3′
5′-GalNAc-YYYAYAXAYXXXGXXGAXYY-3′;
5′-FA-YYYAYAXAYXXXGXXGAXYY-3′;
5′-DCL-YYYAYAXAYXXXGXXGAXYY-3′;
5′-UAMC1110-YYYAYAXAYXXXGXXGAXYY-3′;
3WJ-C with floxuridine and gemcitabine and
derivatives:
5′-GGAXYAAXYAXGGYAA-3′;
(targeting aptamer sequences in lower case);
(SEQ ID NO: 14)
5′-GGAXYAAXYAXGGYAAgccuuaguaacgugcuuugaugucgauucga
caggaggc-3′
(SEQ ID NO: 15)
5′-GGAXYAAXYAXGGYAAGGGACCGAAAAAGACCUGACUUCUAUACUAA
GUCUACGUUUCCC-3′;
(targetinga ptamer sequences in lower case);
(SEQ ID NO: 16)
5′-GGAXYAAXYAXGGYAAgcgacugguuacccggucg-3′
(targeting aptamer sequences in lower case);
(SEQ ID NO: 17)
5′-GGAXYAAXYAXGGYAAcagaacguauacuauucug-3′
(SEQ ID NO: 18)
5′-GalNAc-GGAXYAAXYAXGGYAA-3′;
3WJ-c-FA;
(SEQ ID NO: 18)
5′-FA-GGAXYAAXYAXGGYAA-3′,
(SEQ ID NO: 19)
5′-DCL-GGAXYAAXYAXGGYAA-3′;
(SEQ ID NO: 20)
5′-UAMC1110-GGAXYAAXYAXGGYAA-3′;
4WJ-A with floxuridine and gemcitabine and
derivatives:
(SEQ ID NO: 21)
5′-XXAGGXAAAGYYAYYXGYAGGXGYXAYYGAXGXAAXXYAA-3′;
(targeting aptamer sequences in lower case);
(SEQ ID NO: 22)
5′-XXAGGXAAAGYYAYYXGYAGGXGYXAYYGAXGXAAXXYAAgccuuag
uaacgugcuuugaugucgauucgacaggaggc-3′
(targeting aptamer sequences in lower case);
(SEQ ID NO: 23)
5′-XXAGGXAAAGYYAYYXGYAGGXGYXAYYGAXGXAAXXYAAgggaccg
aaaaagaccugacuucuauacuaagucuacguuuccc-3′
(targeting aptamer sequences in lower case);
(SEQ ID NO: 24)
5′-XXAGGXAAAGYYAYYXGYAGGXGYXAYYGAXGXAAXXYAAgcgacug
guuacccggucg-3′
(targeting aptamer sequences in lower case);
(SEQ ID NO: 25)
5′-XXAGGXAAAGYYAYYXGYAGGXGYXAYYGAXGXAAXXYAAcagaacg
uauacuauucug-3′
(SEQ ID NO: 21)
5′-GalNAc-XXAGGXAAAGYYAYYXGYAGGXGYXAYYGAXGXAAXXYA
A-3′;
(SEQ ID NO: 21)
5′-FA-XXAGGXAAAGYYAYYXGYAGGXGYXAYYGAXGXAAXXYAA-3′;
(SEQ ID NO: 21)
5′-DCL-XXAGGXAAAGYYAYYXGYAGGXGYXAYYGAXGXAAXXYAA-
3′;
(SEQ ID NO: 21)
5′-UAMC1110-XXAGGXAAAGYYAYYXGYAGGXGYXAYYGAXGXAAXXY
AA-3′;
4WJ-B with floxuridine and gemcitabine and
derivatives:
(SEQ ID NO: 26)
5′-XXGAAXXAYAXYGGXAGYAYGGGYXGXGYGAGGYXGAAYAG-3′;
(targeting aptamer sequences in lower case);
(SEQ ID NO: 27)
5′-XXGAAXXAYAXYGGXAGYAYGGGYXGXGYGAGGYXGAAYAGgccuua
guaacgugcuuugaugucgauucgacaggaggc-3′
(targeting aptamer sequences in lower case);
(SEQ ID NO: 28)
5′-XXGAAXXAYAXYGGXAGYAYGGGYXGXGYGAGGYXGAAYAGgggacc
gaaaaagaccugacuucuauacuaagucuacguuuccc-3′
(targeting aptamer sequences in lower case);
(SEQ ID NO: 29)
5′-XXGAAXXAYAXYGGXAGYAYGGGYXGXGYGAGGYXGAAYAGgcgacu
gguuacccggucg-3′
(targeting aptamer sequences in lower case);
(SEQ ID NO: 30)
5′-XXGAAXXAYAXYGGXAGYAYGGGYXGXGYGAGGYXGAAYAGcagaac
guauacuauucug-3′
(SEQ ID NO: 26)
5′-GalNAc-XXGAAXXAYAXYGGXAGYAYGGGYXGXGYGAGGYXGAAYA
G-3′;
(SEQ ID NO: 26)
5′-FA-XXGAAXXAYAXYGGXAGYAYGGGYXGXGYGAGGYXGAAYAG-
3′;
(SEQ ID NO: 26)
5′-DCL-XXGAAXXAYAXYGGXAGYAYGGGYXGXGYGAGGYXGAAYAG-
3′;
(SEQ ID NO: 26)
5′-UAMC1110-XXGAAXXAYAXYGGXAGYAYGGGYXGXGYGAGGYXGAA
YAG-3′;
4WJ-C with floxuridine and gemcitabine and
derivatives:
(SEQ ID NO: 31)
5′-YXGXXYAGYYXYGYAYAGYYAGYAYGYAYYXGAAXAGG-3′;
(targeting aptamer sequences in lower case);
(SEQ ID NO: 32)
5′-YXGXXYAGYYXYGYAYAGYYAGYAYGYAYYXGAAXAGGgccuuagua
acgugcuuugaugucgauucgacaggaggc-3′
(targeting aptamer sequences in lower case);
(SEQ ID NO: 33)
5′-YXGXXYAGYYXYGYAYAGYYAGYAYGYAYYXGAAXAGGgggaccgaa
aaagaccugacuucuauacuaagucuacguuuccc-3′
(targeting aptamer sequences in lower case);
(SEQ ID NO: 34)
5′-YXGXXYAGYYXYGYAYAGYYAGYAYGYAYYXGAAXAGGgcgacuggu
uacccggucg-3′
(targeting aptamer sequences in lower case);
(SEQ ID NO: 35)
5′-YXGXXYAGYYXYGYAYAGYYAGYAYGYAYYXGAAXAGGcagaacgua
uacuauucug-3′
(SEQ ID NO: 31)
5′-GalNAc-YXGXXYAGYYXYGYAYAGYYAGYAYGYAYYXGAAXAGG-
3′;
(SEQ ID NO: 31)
5′-FA-YXGXXYAGYYXYGYAYAGYYAGYAYGYAYYXGAAXAGG-3′;
(SEQ ID NO: 31)
5′-DCL-YXGXXYAGYYXYGYAYAGYYAGYAYGYAYYXGAAXAGG-3′;
(SEQ ID NO: 31)
5′-UAMC1110-YXGXXYAGYYXYGYAYAGYYAGYAYGYAYYXGAAXAG
G-3′;
4WJ-D with floxuridine and gemcitabine and
derivatives:
(SEQ ID NO: 36)
5′-YYXAXXYAGGXGYGXGYXGGGYXGYAGGXGGYXXXAYYXAA-3′;
(targeting aptamer sequences in lower case);
(SEQ ID NO: 37)
5′-YYXAXXYAGGXGYGXGYXGGGYXGYAGGXGGYXXXAYYXAAgccuua
guaacgugcuuugaugucgauucgacaggaggc-3′
(targeting aptamer sequences in lower case);
(SEQ ID NO: 38)
5′-YYXAXXYAGGXGYGXGYXGGGYXGYAGGXGGYXXXAYYXAAgggacc
gaaaaagaccugacuucuauacuaagucuacguuuccc-3′
(targeting aptamer sequences in lower case);
(SEQ ID NO: 39)
5′-YYXAXXYAGGXGYGXGYXGGGYXGYAGGXGGYXXXAYYXAAgcgacu
gguuacccggucg-3′
(targeting aptamer sequences in lower case);
(SEQ ID NO: 40)
5′-YYXAXXYAGGXGYGXGYXGGGYXGYAGGXGGYXXXAYYXAAcagaac
guauacuauucug-3′
(SEQ ID NO: 36)
5′-GalNAc-YYXAXXYAGGXGYGXGYXGGGYXGYAGGXGGYXXXAYYXA
A-3′;
(SEQ ID NO: 36)
5′-FA-YYXAXXYAGGXGYGXGYXGGGYXGYAGGXGGYXXXAYYXAA-
3′;
(SEQ ID NO: 36)
5′-DCL-YYXAXXYAGGXGYGXGYXGGGYXGYAGGXGGYXXXAYYXAA-
3′;
(SEQ ID NO: 36)
5′-UAMC1110-YYXAXXYAGGXGYGXGYXGGGYXGYAGGXGGYXXXAYY
XAA-3′;
6WJ-A with floxuridine and gemcitabine and
derivatives:
(SEQ ID NO: 41)
5′-GAGXAXAXGXXAGGYYXGGGXGAGXYYXXGYGXYXXYXAYYG-3′;
(targeting aptamer sequences in lower case);
(SEQ ID NO: 42)
5′-GAGXAXAXGXXAGGYYXGGGXGAGXYYXXGYGXYXXYXAYYGgccuu
aguaacgugcuuugaugucgauucgacaggaggc-3′
(targeting aptamer sequences in lower case);
(SEQ ID NO: 43)
5′-GAGXAXAXGXXAGGYYXGGGXGAGXYYXXGYGXYXXYXAYYGgggac
cgaaaaagaccugacuucuauacuaagucuacguuuccc-3′
(targeting aptamer sequences in lower case);
(SEQ ID NO: 44)
5′-GAGXAXAXGXXAGGYYXGGGXGAGXYYXXGYGXYXXYXAYYGgcgac
ugguuacccggucg-3′
(targeting aptamer sequences in lower case);
(SEQ ID NO: 45)
5′-GAGXAXAXGXXAGGYYXGGGXGAGXYYXXGYGXYXXYXAYYGcagaa
cquauacuauucug-3′
(SEQ ID NO: 41)
5′-GalNAc-GAGXAXAXGXXAGGYYXGGGXGAGXYYXXGYGXYXXYXAY
YG-3′;
(SEQ ID NO: 41)
5′-FA-GAGXAXAXGXXAGGYYXGGGXGAGXYYXXGYGXYXXYXAYYG-
3′;
(SEQ ID NO: 41)
5′-DCL-GAGXAXAXGXXAGGYYXGGGXGAGXYYXXGYGXYXXYXAYYG-
3′;
(SEQ ID NO: 41)
5′-UAMC1110-GAGXAXAXGXXAGGYYXGGGXGAGXYYXXGYGXYXXYX
AYYG-3′;
6WJ-B with floxuridine and gemcitabine and
derivatives:
(SEQ ID NO: 46)
5′-YGGXAGAAGAYGYAAGGAYXXGYXAGXXGXGGXAYXGXXYYY-3′;
(targeting aptamer sequences in lower case);
(SEQ ID NO: 47)
5′-YGGXAGAAGAYGYAAGGAYXXGYXAGXXGXGGXAYXGXXYYYgccuu
aguaacgugcuuugaugucgauucgacaggaggc-3′
(targeting aptamer sequences in lower case);
(SEQ ID NO: 48)
5′-YGGXAGAAGAYGYAAGGAYXXGYXAGXXGXGGXAYXGXXYYYgggac
cgaaaaagaccugacuucuauacuaagucuacguuuccc-3′
(targeting aptamer sequences in lower case);
(SEQ ID NO: 49)
5′-YGGXAGAAGAYGYAAGGAYXXGYXAGXXGXGGXAYXGXXYYYgcgac
ugguuacccggucg-3′
(targeting aptamer sequences in lower case);
(SEQ ID NO: 50)
5′-YGGXAGAAGAYGYAAGGAYXXGYXAGXXGXGGXAYXGXXYYYcagaa
cguauacuauucug-3′
(SEQ ID NO: 46)
5′-GalNAc-YGGXAGAAGAYGYAAGGAYXXGYXAGXXGXGGXAYXGXXY
YY-3′;
(SEQ ID NO: 46)
5′-FA-YGGXAGAAGAYGYAAGGAYXXGYXAGXXGXGGXAYXGXXYYY-
3′;
(SEQ ID NO: 46)
5′-DCL-YGGXAGAAGAYGYAAGGAYXXGYXAGXXGXGGXAYXGXXYYY-
3′;
(SEQ ID NO: 46)
5′-UAMC1110-YGGXAGAAGAYGYAAGGAYXXGYXAGXXGXGGXAYXGX
XYYY-3′;
6WJ-C with floxuridine and gemcitabine and
derivatives:
(SEQ ID NO: 51)
5′-GGGAAYAGXAYYAYAAYXAGXGXYYYGGGAXAGGGAYAXAYA-3′;
(targeting aptamer sequences in lower case);
(SEQ ID NO: 52)
5′-GGGAAYAGXAYYAYAAYXAGXGXYYYGGGAXAGGGAYAXAYAgccuu
aguaacgugcuuugaugucgauucgacaggaggc-3′
(targeting aptamer sequences in lower case);
(SEQ ID NO: 53)
5′-GGGAAYAGXAYYAYAAYXAGXGXYYYGGGAXAGGGAYAXAYAgggac
cgaaaaagaccugacuucuauacuaagucuacguuuccc-3′
(targeting aptamer sequences in lower case);
(SEQ ID NO: 54)
5′-GGGAAYAGXAYYAYAAYXAGXGXYYYGGGAXAGGGAYAXAYAgcgac
ugguuacccggucg-3′
(targeting aptamer sequences in lower case);
(SEQ ID NO: 55)
5′-GGGAAYAGXAYYAYAAYXAGXGXYYYGGGAXAGGGAYAXAYAcagaa
cguauacuauucug-3′
(SEQ ID NO: 51)
5′-GalNAc-GGGAAYAGXAYYAYAAYXAGXGXYYYGGGAXAGGGAYAXA
YA-3′;
(SEQ ID NO: 51)
5′-FA-GGGAAYAGXAYYAYAAYXAGXGXYYYGGGAXAGGGAYAXAYA-
3′;
(SEQ ID NO: 51)
5′-DCL-GGGAAYAGXAYYAYAAYXAGXGXYYYGGGAXAGGGAYAXAYA-
3′;
(SEQ ID NO: 51)
5′-UAMC1110-GGGAAYAGXAYYAYAAYXAGXGXYYYGGGAXAGGGAYA
XAYA-3′;
6WJ-D with floxuridine and gemcitabine and
derivatives:
(SEQ ID NO: 56)
5′-XGXAXGXYYYXAXYYYGGGAXGYYYAGGYYXAAYAXAXAYXY-3′;
(targeting aptamer sequences in lower case);
(SEQ ID NO: 57)
5′-XGXAXGXYYYXAXYYYGGGAXGYYYAGGYYXAAYAXAXAYXYgccuu
aguaacgugcuuugaugucgauucgacaggaggc-3′
(targeting aptamer sequences in lower case);
(SEQ ID NO: 58)
5′-XGXAXGXYYYXAXYYYGGGAXGYYYAGGYYXAAYAXAXAYXYgggac
cgaaaaagaccugacuucuauacuaagucuacguuuccc-3′
(targeting aptamer sequences in lower case);
(SEQ ID NO: 59)
5′-XGXAXGXYYYXAXYYYGGGAXGYYYAGGYYXAAYAXAXAYXYgcgac
ugguuacccggucg-3′
(targeting aptamer sequences in lower case);
(SEQ ID NO: 60)
5′-XGXAXGXYYYXAXYYYGGGAXGYYYAGGYYXAAYAXAXAYXYcagaa
cguauacuauucug-3′
(SEQ ID NO: 56)
5′-GalNAc-XGXAXGXYYYXAXYYYGGGAXGYYYAGGYYXAAYAXAXAY
XY-3′;
(SEQ ID NO: 56)
5′-FA-XGXAXGXYYYXAXYYYGGGAXGYYYAGGYYXAAYAXAXAYXY-
3′;
(SEQ ID NO: 56)
5′-DCL-XGXAXGXYYYXAXYYYGGGAXGYYYAGGYYXAAYAXAXAYXY-
3′;
or
(SEQ ID NO: 56)
5′-UAMC1110-XGXAXGXYYYXAXYYYGGGAXGYYYAGGYYXAAYAXAX
AYXY-3′, wherein X is floxuridine(5-fluorodeoxy-
uridine, UB5F), and wherein Y is gemcitabine(2′,
2′-difluoro-2′-deoxycytidine, dFdC, CR2FF).
In some embodiments, the disclosed RNA
oligonucleotide has one of the following
sequences:
3WJ-A with only gemcitabine and derivatives:
(SEQ ID NO: 61)
5′-UUGYYAUGUGUAUGUGGG-3′;
(targeting aptamer sequences in lower case);
(SEQ ID NO: 280)
5′-UUGYYAUGUGUAUGUGGGGgccuuaguaacgugcuuugaugucgauu
cgacaggaggc-3′
(SEQ ID NO: 281)
5′-UUGYYAUGUGUAUGUGGGGGGACCGAAAAAGACCUGACUUCUAUACU
AAGUCUACGUUUCCC-3′;
(targeting aptamer sequences in lower case);
(SEQ ID NO: 282)
5′-UUGYYAUGUGUAUGUGGGgcgacugguuacccggucg-3′
(targeting aptamer sequences in lower case);
(SEQ ID NO: 283)
5′-UUGYYAUGUGUAUGUGGGcagaacguauacuauucug-3′
(SEQ ID NO: 61)
5′-GalNAc-UUGYYAUGUGUAUGUGGG-3′;
3WJ a FA;
(SEQ ID NO: 61)
5′-FA-UUGYYAUGUGUAUGUGGG-3′,
(SEQ ID NO: 61)
5′-DCL-UUGYYAUGUGUAUGUGGG-3′;
(SEQ ID NO: 61)
5′-UAMC1110-UUGYYAUGUGUAUGUGGG-3′;
3WJ-B with only gemcitabine and derivatives:
(SEQ ID NO: 62)
5′-YYYAYAUAYUUUGUUGAUYY-3′;
(targeting aptamer sequences in lower case);
(SEQ ID NO: 63)
5′-YYYAYAUAYUUUGUUGAUYYgccuuaguaacgugcuuugaugucgau
ucgacaggaggc-3′
(SEQ ID NO: 64)
5′-YYYAYAUAYUUUGUUGAUYYGGGACCGAAAAAGACCUGACUUCUAUA
CUAAGUCUACGUUUCCC-3′;
3WJ-b-EpCAMapt
(targeting aptamer sequences in lower case);
(SEQ ID NO: 65)
5′-YYYAYAUAYUUUGUUGAUYYgcgacugguuacccggucg-3′,
(targeting aptamer sequences in lower case);
(SEQ ID NO: 66)
5′-YYYAYAUAYUUUGUUGAUYYcagaacguauacuauucug-3′
(SEQ ID NO: 62)
5′-GalNAc-YYYAYAUAYUUUGUUGAUYY-3′;
(SEQ ID NO: 62)
5′-FA-YYYAYAUAYUUUGUUGAUYY-3′;
(SEQ ID NO: 62)
5′-DCL-YYYAYAUAYUUUGUUGAUYY-3′;
(SEQ ID NO: 62)
5′-UAMC1110-YYYAYAUAYUUUGUUGAUYY-3′;
3WJ-C with only gemcitabine and derivatives:
(SEQ ID NO: 67)
5′-GGAUYAAUYAUGGYAA-3′;
(targeting aptamer sequences in lower case)
(SEQ ID NO: 68)
5′-GGAUYAAUYAUGGYAAgccuuaguaacgugcuuugaugucgauucga
caggaggc-3′;
(SEQ ID NO: 69)
5′-GGAUYAAUYAUGGYAAGGGACCGAAAAAGACCUGACUUCUAUACUAA
GUCUACGUUUCCC-3′;
(targeting aptamer sequences in lower case);
(SEQ ID NO: 70)
5′-GGAUYAAUYAUGGYAAgcgacugguuacccggucg-3′
(targeting aptamer sequences in lower case);
(SEQ ID NO: 71)
5′-GGAUYAAUYAUGGYAAcagaacguauacuauucug-3′
(SEQ ID NO: 67)
5′-GalNAc-GGAUYAAUYAUGGYAA-3′;
(SEQ ID NO: 67)
5′-FA-GGAUYAAUYAUGGYAA-3′;
(SEQ ID NO: 67)
5′-DCL-GGAUYAAUYAUGGYAA-3′;
(SEQ ID NO: 67)
5′-UAMC1110-GGAUYAAUYAUGGYAA-3′;
4WJ-A with only gemcitabine and derivatives:
(SEQ ID NO: 72)
5′-UUAGGUAAAGYYAYYUGYAGGUGYUAYYGAUGUAAUUYAA-3′;
(targeting aptamer sequences in lower case);
(SEQ ID NO: 73)
5′-UUAGGUAAAGYYAYYUGYAGGUGYUAYYGAUGUAAUUYAAgccuuag
uaacgugcuuugaugucgauucgacaggaggc-3′
(targeting aptamer sequences in lower case);
(SEQ ID NO: 74)
5′-UUAGGUAAAGYYAYYUGYAGGUGYUAYYGAUGUAAUUYAAgggaccg
aaaaagaccugacuucuauacuaagucuacguuuccc-3′
(targeting aptamer sequences in lower case);
(SEQ ID NO: 75)
5′-UUAGGUAAAGYYAYYUGYAGGUGYUAYYGAUGUAAUUYAAgcgacug
guuacccggucg-3′
(targeting aptamer sequences in lower case);
(SEQ ID NO: 76)
5′-UUAGGUAAAGYYAYYUGYAGGUGYUAYYGAUGUAAUUYAAcagaacg
uauacuauucug-3′
(SEQ ID NO: 72)
5′-GalNAc-UUAGGUAAAGYYAYYUGYAGGUGYUAYYGAUGUAAUUYA
A-3′;
(SEQ ID NO: 72)
5′-FA-UUAGGUAAAGYYAYYUGYAGGUGYUAYYGAUGUAAUUYAA-3′;
(SEQ ID NO: 72)
5′-DCL-UUAGGUAAAGYYAYYUGYAGGUGYUAYYGAUGUAAUUYAA-
3′;
(SEQ ID NO: 72)
5′-UAMC1110-UUAGGUAAAGYYAYYUGYAGGUGYUAYYGAUGUAAUUY
AA-3′;
4WJ-B with only gemcitabine and derivatives:
(SEQ ID NO: 77)
5′-UUGAAUUAYAUYGGUAGYAYGGGYUGUGYGAGGYUGAAYAG-3′;
(targeting aptamer sequences in lower case);
(SEQ ID NO: 78)
5′-UUGAAUUAYAUYGGUAGYAYGGGYUGUGYGAGGYUGAAYAGgccuua
guaacgugcuuugaugucgauucgacaggaggc-3′
(targeting aptamer sequences in lower case);
(SEQ ID NO: 79)
5′-UUGAAUUAYAUYGGUAGYAYGGGYUGUGYGAGGYUGAAYAGgggacc
gaaaaagaccugacuucuauacuaagucuacguuuccc-3′
(targeting aptamer sequences in lower case);
(SEQ ID NO: 80)
5′-UUGAAUUAYAUYGGUAGYAYGGGYUGUGYGAGGYUGAAYAGgcgacu
gguuacccggucg-3′
(targeting aptamer sequences in lower case);
(SEQ ID NO: 81)
5′-UUGAAUUAYAUYGGUAGYAYGGGYUGUGYGAGGYUGAAYAGcagaac
guauacuauucug-3′
(SEQ ID NO: 77)
5′-GalNAc-UUGAAUUAYAUYGGUAGYAYGGGYUGUGYGAGGYUGAAYA
G-3′;
(SEQ ID NO: 77)
5′-FA-UUGAAUUAYAUYGGUAGYAYGGGYUGUGYGAGGYUGAAYAG-
3′;
(SEQ ID NO: 77)
5′-DCL-UUGAAUUAYAUYGGUAGYAYGGGYUGUGYGAGGYUGAAYAG-
3′;
(SEQ ID NO: 77)
5′-UAMC1110-UUGAAUUAYAUYGGUAGYAYGGGYUGUGYGAGGYUGAA
YAG-3′;
4WJ-C with only gemcitabine and derivatives:
(SEQ ID NO: 82)
5′-YUGUUYAGYYUYGYAYAGYYAGYAYGYAYYUGAAUAGG-3′;
(targeting aptamer sequences in lower case);
(SEQ ID NO: 83)
5′-YUGUUYAGYYUYGYAYAGYYAGYAYGYAYYUGAAUAGGgccuuagua
acgugcuuugaugucgauucgacaggaggc-3′
(targeting aptamer sequences in lower case);
(SEQ ID NO: 84)
5′-YUGUUYAGYYUYGYAYAGYYAGYAYGYAYYUGAAUAGGgggaccgaa
aaagaccugacuucuauacuaagucuacguuuccc-3′
(targeting aptamer sequences in lower case);
(SEQ ID NO: 85)
5′-YUGUUYAGYYUYGYAYAGYYAGYAYGYAYYUGAAUAGGgcgacuggu
uacccggucg-3′
(targeting aptamer sequences in lower case);
(SEQ ID NO: 86)
5′-YUGUUYAGYYUYGYAYAGYYAGYAYGYAYYUGAAUAGGcagaacgua
uacuauucug-3′
(SEQ ID NO: 82)
5′-GalNAc-YUGUUYAGYYUYGYAYAGYYAGYAYGYAYYUGAAUAGG-
3′;
(SEQ ID NO: 82)
5′-FA-YUGUUYAGYYUYGYAYAGYYAGYAYGYAYYUGAAUAGG-3′;
(SEQ ID NO: 82)
5′-DCL-YUGUUYAGYYUYGYAYAGYYAGYAYGYAYYUGAAUAGG-3′;
(SEQ ID NO: 82)
5′-UAMC1110-YUGUUYAGYYUYGYAYAGYYAGYAYGYAYYUGAAUAG
G-3′;
4WJ-D with only gemcitabine and derivatives:
(SEQ ID NO: 87)
5′-YYUAUUYAGGUGYGUGYUGGGYUGYAGGUGGYUUUAYYUAA-3′;
(targeting aptamer sequences in lower case);
(SEQ ID NO: 88)
5′-YYUAUUYAGGUGYGUGYUGGGYUGYAGGUGGYUUUAYYUAAgccuua
guaacgugcuuugaugucgauucgacaggaggc-3′
(targeting aptamer sequences in lower case);
(SEQ ID NO: 89)
5′-YYUAUUYAGGUGYGUGYUGGGYUGYAGGUGGYUUUAYYUAAgggacc
gaaaaagaccugacuucuauacuaagucuacguuuccc-3′
(targeting aptamer sequences in lower case);
(SEQ ID NO: 90)
5′-YYUAUUYAGGUGYGUGYUGGGYUGYAGGUGGYUUUAYYUAAgcgacu
gguuacccggucg-3′
(targeting aptamer sequences in lower case);
(SEQ ID NO: 91)
5′-YYUAUUYAGGUGYGUGYUGGGYUGYAGGUGGYUUUAYYUAAcagaac
guauacuauucug-3′
(SEQ ID NO: 87)
5′-GalNAc-YYUAUUYAGGUGYGUGYUGGGYUGYAGGUGGYUUUAYYUA
A-3′;
(SEQ ID NO: 87)
5′-FA-YYUAUUYAGGUGYGUGYUGGGYUGYAGGUGGYUUUAYYUAA-
3′;
(SEQ ID NO: 87)
5′-DCL-YYUAUUYAGGUGYGUGYUGGGYUGYAGGUGGYUUUAYYUAA-
3′;
(SEQ ID NO: 87)
5′-UAMC1110-YYUAUUYAGGUGYGUGYUGGGYUGYAGGUGGYUUUAYY
UAA-3′;
6WJ-A with only gemcitabine and derivatives:
(SEQ ID NO: 92)
5′-GAGUAUAUGUUAGGYYUGGGUGAGUYYUUGYGUYUUYUAYYG-3′;
(targeting aptamer sequences in lower case);
(SEQ ID NO: 93)
5′-GAGUAUAUGUUAGGYYUGGGUGAGUYYUUGYGUYUUYUAYYGgccuu
aguaacgugcuuugaugucgauucgacaggaggc-3′
(targeting aptamer sequences in lower case);
(SEQ ID NO: 94)
5′-GAGUAUAUGUUAGGYYUGGGUGAGUYYUUGYGUYUUYUAYYGgggac
cgaaaaagaccugacuucuauacuaagucuacguuuccc-3′
(targeting aptamer sequences in lower case);
(SEQ ID NO: 95)
5′-GAGUAUAUGUUAGGYYUGGGUGAGUYYUUGYGUYUUYUAYYGgcgac
ugguuacccggucg-3′
(targeting aptamer sequences in lower case);
(SEQ ID NO: 96)
5′-GAGUAUAUGUUAGGYYUGGGUGAGUYYUUGYGUYUUYUAYYGcagaa
cguauacuauucug-3′
(SEQ ID NO: 92)
5′-GalNAc-GAGUAUAUGUUAGGYYUGGGUGAGUYYUUGYGUYUUYUAY
YG-3′;
(SEQ ID NO: 92)
5′-FA-GAGUAUAUGUUAGGYYUGGGUGAGUYYUUGYGUYUUYUAYYG-
3′;
(SEQ ID NO: 92)
5′-DCL-GAGUAUAUGUUAGGYYUGGGUGAGUYYUUGYGUYUUYUAYYG-
3′;
(SEQ ID NO: 92)
5′-UAMC1110-GAGUAUAUGUUAGGYYUGGGUGAGUYYUUGYGUYUUYU
AYYG-3′;
6WJ-B with only gemcitabine and derivatives:
(SEQ ID NO: 97)
5′-YGGUAGAAGAYGYAAGGAYUUGYUAGUUGUGGUAYUGUUYYY-3′;
(targeting aptamer sequences in lower case);
(SEQ ID NO: 98)
5′-YGGUAGAAGAYGYAAGGAYUUGYUAGUUGUGGUAYUGUUYYYgccuu
aguaacgugcuuugaugucgauucgacaggaggc-3′
(targeting aptamer sequences in lower case);
(SEQ ID NO: 99)
5′-YGGUAGAAGAYGYAAGGAYUUGYUAGUUGUGGUAYUGUUYYYgggac
cgaaaaagaccugacuucuauacuaagucuacguuuccc-3′
(targeting aptamer sequences in lower case);
(SEQ ID NO: 100)
5′-YGGUAGAAGAYGYAAGGAYUUGYUAGUUGUGGUAYUGUUYYYgcgac
ugguuacccggucg-3′
(targeting aptamer sequences in lower case);
(SEQ ID NO: 101)
5′-YGGUAGAAGAYGYAAGGAYUUGYUAGUUGUGGUAYUGUUYYYcagaa
cguauacuauucug-3′
(SEQ ID NO: 97)
5′-GalNAc-YGGUAGAAGAYGYAAGGAYUUGYUAGUUGUGGUAYUGUUY
YY-3;
(SEQ ID NO: 97)
5′-FA-YGGUAGAAGAYGYAAGGAYUUGYUAGUUGUGGUAYUGUUYYY-
3′;
(SEQ ID NO: 97)
5′-DCL-YGGUAGAAGAYGYAAGGAYUUGYUAGUUGUGGUAYUGUUYYY-
3′;
(SEQ ID NO: 97)
5′-UAMC1110-YGGUAGAAGAYGYAAGGAYUUGYUAGUUGUGGUAYUGU
UYYY-3′;
6WJ-C with only gemcitabine and derivatives:
(SEQ ID NO: 102)
5′-GGGAAYAGUAYYAYAAYUAGUGUYYYGGGAUAGGGAYAUAYA-3′;
(targeting aptamer sequences in lower case);
(SEQ ID NO: 103)
5′-GGGAAYAGUAYYAYAAYUAGUGUYYYGGGAUAGGGAYAUAYAgccuu
aguaacgugcuuugaugucgauucgacaggaggc-3′
(targeting aptamer sequences in lower case);
(SEQ ID NO: 104)
5′-GGGAAYAGUAYYAYAAYUAGUGUYYYGGGAUAGGGAYAUAYAgggac
cgaaaaagaccugacuucuauacuaagucuacguuuccc-3′
(targeting aptamer sequences in lower case);
(SEQ ID NO: 105)
5′-GGGAAYAGUAYYAYAAYUAGUGUYYYGGGAUAGGGAYAUAYAgcgac
ugguuacccggucg-3′
(targeting aptamer sequences in lower case);
(SEQ ID NO: 106)
5′-GGGAAYAGUAYYAYAAYUAGUGUYYYGGGAUAGGGAYAUAYAcagaa
cquauacuauucug-3′
(SEQ ID NO: 102)
5′-GalNAc-GGGAAYAGUAYYAYAAYUAGUGUYYYGGGAUAGGGAYAUA
YA-3′;
(SEQ ID NO: 102)
5′-FA-GGGAAYAGUAYYAYAAYUAGUGUYYYGGGAUAGGGAYAUAYA-
3′;
(SEQ ID NO: 102)
5′-DCL-GGGAAYAGUAYYAYAAYUAGUGUYYYGGGAUAGGGAYAUAYA-
3′;
(SEQ ID NO: 102)
5′-UAMC1110-GGGAAYAGUAYYAYAAYUAGUGUYYYGGGAUAGGGAYA
UAYA-3′;
6WJ-D with only gemcitabine and derivatives:
(SEQ ID NO: 107)
5′-UGUAUGUYYYUAUYYYGGGAUGYYYAGGYYUAAYAUAUAYUY-3′;
(targeting aptamer sequences in lower case);
(SEQ ID NO: 108)
5′-UGUAUGUYYYUAUYYYGGGAUGYYYAGGYYUAAYAUAUAYUYgccuu
aguaacgugcuuugaugucgauucgacaggaggc-3′
(targeting aptamer sequences in lower case);
(SEQ ID NO: 109)
5′-UGUAUGUYYYUAUYYYGGGAUGYYYAGGYYUAAYAUAUAYUYgggac
cgaaaaagaccugacuucuauacuaagucuacguuuccc-3′
(targeting aptamer sequences in lower case);
(SEQ ID NO: 110)
5′-UGUAUGUYYYUAUYYYGGGAUGYYYAGGYYUAAYAUAUAYUYgcgac
ugguuacccggucg-3′
(targeting aptamer sequences in lower case);
(SEQ ID NO: 111)
5′-UGUAUGUYYYUAUYYYGGGAUGYYYAGGYYUAAYAUAUAYUYcagaa
cguauacuauucug-3′
(SEQ ID NO: 107)
5′-GalNAc-UGUAUGUYYYUAUYYYGGGAUGYYYAGGYYUAAYAUAUAY
UY-3′;
(SEQ ID NO: 107)
5′-FA-UGUAUGUYYYUAUYYYGGGAUGYYYAGGYYUAAYAUAUAYUY-
3′;
(SEQ ID NO: 107)
5′-DCL-UGUAUGUYYYUAUYYYGGGAUGYYYAGGYYUAAYAUAUAYUY-
3′;
or
(SEQ ID NO: 107)
5′-UAMC1110-UGUAUGUYYYUAUYYYGGGAUGYYYAGGYYUAAYAUAU
AYUY-3′, wherein Y is gemcitabine(2′, 2′-difluoro-
2′-deoxycytidine, dFdC, CR2FF).
In some embodiments, the disclosed RNA
oligonucleotide has one of the following
sequences:
3WJ-A with floxuridine only and derivatives:
(SEQ ID NO: 112)
5′-XXGCCAXGXGXAXGXGGG-3′;
(targeting aptamer sequences in lower case);
(SEQ ID NO: 113)
5′-XXGCCAXGXGXAXGXGGGGgccuuaguaacgugcuuugaugucgauu
cgacaggaggc-3′
(targeting aptamer sequences in lower case);
(SEQ ID NO: 114)
5′-XXGCCAXGXGXAXGXGGGgggaccgaaaaagaccugacuucuauacu
aagucuacguuuccc-3′
(targeting aptamer sequences in lower case);
(SEQ ID NO: 115)
5′-XXGCCAXGXGXAXGXGGGgcgacugguuacccggucg-3′
3WJ-a-CD133apt
(targeting aptamer sequences in lower case);
(SEQ ID NO: 116)
5′-XXGCCAXGXGXAXGXGGGcagaacguauacuauucug-3′,
(SEQ ID NO: 112)
5′-GalNAc-XXGCCAXGXGXAXGXGGG-3′;
(SEQ ID NO: 112)
5′-FA-XXGCCAXGXGXAXGXGGG-3′;
(SEQ ID NO: 112)
5′-DCL-XXGCCAXGXGXAXGXGGG-3′;
(SEQ ID NO: 112)
5′-UAMC1110-XXGCCAXGXGXAXGXGGG-3′;
3WJ-B with floxuridine only and derivatives:
3WJ-b;
(SEQ ID NO: 117)
5′-CCCACAXACXXXGXXGAXCC-3′,
(targeting aptamer sequences in lower case);
(SEQ ID NO: 118)
5′-CCCACAXACXXXGXXGAXCCgccuuaguaacgugcuuugaugucgau
ucgacaggaggc-3′
(targeting aptamer sequences in lower case);
(SEQ ID NO: 119)
5′-CCCACAXACXXXGXXGAXCCgggaccgaaaaagaccugacuucuaua
cuaagucuacguuuccc-3′
(targeting aptamer sequences in lower case);
(SEQ ID NO: 120)
5′-CCCACAXACXXXGXXGAXCCgcgacugguuacccggucg-3′
(targeting aptamer sequences in lower case);
(SEQ ID NO: 121)
5′-CCCACAXACXXXGXXGAXCCcagaacguauacuauucug-3′
(SEQ ID NO: 117)
5′-GalNAc-CCCACAXACXXXGXXGAXCC-3′;
(SEQ ID NO: 117)
5′-FA-CCCACAXACXXXGXXGAXCC-3′;
(SEQ ID NO: 117)
5′-DCL-CCCACAXACXXXGXXGAXCC-3′;
SEQ ID NO: 117)
5′-UAMC1110-CCCACAXACXXXGXXGAXCC-3′;
3WJ-C with floxuridine only and derivatives:
(SEQ ID NO: 122)
5′-GGAXCAAXCAXGGCAA-3′;
(targeting aptamer sequences in lower case);
(SEQ ID NO: 123)
5′-GGAXCAAXCAXGGCAAgccuuaguaacgugcuuugaugucgauucga
caggaggc-3′
(targeting aptamer sequences in lower case);
(SEQ ID NO: 124)
5′-GGAXCAAXCAXGGCAAgggaccgaaaaagaccugacuucuauacuaa
gucuacguuuccc-3′
(targeting aptamer sequences in lower case);
(SEQ ID NO: 125)
5′-GGAXCAAXCAXGGCAAgcgacugguuacccggucg-3′
(targeting aptamer sequences in lower case);
(SEQ ID NO: 126)
5′-GGAXCAAXCAXGGCAAcagaacguauacuauucug-3′
(SEQ ID NO: 122)
5′-GalNAc-GGAXCAAXCAXGGCAA-3′;
(SEQ ID NO: 122)
5′-FA-GGAXCAAXCAXGGCAA-3′;
(SEQ ID NO: 122)
5′-DCL-GGAXCAAXCAXGGCAA-3′;
(SEQ ID NO: 122)
5′-UAMC1110-GGAXCAAXCAXGGCAA-3′;
4WJ-A with floxuridine only and derivatives:
(SEQ ID NO: 127)
5′-XXAGGXAAAGCCACCXGCAGGXGCXACCGAXGXAAXXCAA-3′;
(targeting aptamer sequences in lower case);
(SEQ ID NO: 128)
5′-XXAGGXAAAGCCACCXGCAGGXGCXACCGAXGXAAXXCAAgccuuag
uaacgugcuuugaugucgauucgacaggaggc-3′
(targeting aptamer sequences in lower case);
(SEQ ID NO: 129)
5′-XXAGGXAAAGCCACCXGCAGGXGCXACCGAXGXAAXXCAAgggaccg
aaaaagaccugacuucuauacuaagucuacguuuccc-3′
(targeting aptamer sequences in lower case);
(SEQ ID NO: 130)
5′-XXAGGXAAAGCCACCXGCAGGXGCXACCGAXGXAAXXCAAgcgacug
guuacccggucg-3′
(targeting aptamer sequences in lower case);
(SEQ ID NO: 131)
5′-XXAGGXAAAGCCACCXGCAGGXGCXACCGAXGXAAXXCAAcagaacg
uauacuauucug-3′
(SEQ ID NO: 127)
5′-GalNAc-XXAGGXAAAGCCACCXGCAGGXGCXACCGAXGXAAXXCA
A-3′;
(SEQ ID NO: 127)
5′-FA-XXAGGXAAAGCCACCXGCAGGXGCXACCGAXGXAAXXCAA-3′;
(SEQ ID NO: 127)
5′-DCL-XXAGGXAAAGCCACCXGCAGGXGCXACCGAXGXAAXXCAA-
3′;
(SEQ ID NO: 127)
5′-UAMC1110-XXAGGXAAAGCCACCXGCAGGXGCXACCGAXGXAAXXC
AA-3′;
4WJ-B with floxuridine only and derivatives:
(SEQ ID NO: 132)
5′-XXGAAXXACAXCGGXAGCACGGGCXGXGCGAGGCXGAACAG-3′;
(targeting aptamer sequences in lower case);
(SEQ ID NO: 133)
5′-XXGAAXXACAXCGGXAGCACGGGCXGXGCGAGGCXGAACAGgccuua
guaacgugcuuugaugucgauucgacaggaggc-3′
(targeting aptamer sequences in lower case);
(SEQ ID NO: 134)
5′-XXGAAXXACAXCGGXAGCACGGGCXGXGCGAGGCXGAACAGgggacc
gaaaaagaccugacuucuauacuaagucuacguuuccc-3′
(targeting aptamer sequences in lower case);
(SEQ ID NO: 135)
5′-XXGAAXXACAXCGGXAGCACGGGCXGXGCGAGGCXGAACAGgcgacu
gguuacccggucg-3′
(targeting aptamer sequences in lower case);
(SEQ ID NO: 136)
5′-XXGAAXXACAXCGGXAGCACGGGCXGXGCGAGGCXGAACAGcagaac
guauacuauucug-3′
(SEQ ID NO: 132)
5′-GalNAc-XXGAAXXACAXCGGXAGCACGGGCXGXGCGAGGCXGAACA
G-3′;
(SEQ ID NO: 132)
5′-FA-XXGAAXXACAXCGGXAGCACGGGCXGXGCGAGGCXGAACAG-
3′;
(SEQ ID NO: 132)
5′-DCL-XXGAAXXACAXCGGXAGCACGGGCXGXGCGAGGCXGAACAG-
3′;
(SEQ ID NO: 132)
5′-UAMC1110-XXGAAXXACAXCGGXAGCACGGGCXGXGCGAGGCXGAA
CAG-3′;
4WJ-C with floxuridine only and derivatives:
(SEQ ID NO: 137)
5′-CXGXXCAGCCXCGCACAGCCAGCACGCACCXGAAXAGG-3′;
(targeting aptamer sequences in lower case);
(SEQ ID NO: 138)
5′-CXGXXCAGCCXCGCACAGCCAGCACGCACCXGAAXAGGgccuuagua
acgugcuuugaugucgauucgacaggaggc-3′
(targeting aptamer sequences in lower case);
(SEQ ID NO: 139)
5′-CXGXXCAGCCXCGCACAGCCAGCACGCACCXGAAXAGGgggaccgaa
aaagaccugacuucuauacuaagucuacguuuccc-3′
(targeting aptamer sequences in lower case);
(SEQ ID NO: 140)
5′-CXGXXCAGCCXCGCACAGCCAGCACGCACCXGAAXAGGgcgacuggu
uacccggucg-3′
(targeting aptamer sequences in lower case);
(SEQ ID NO: 141)
5′-CXGXXCAGCCXCGCACAGCCAGCACGCACCXGAAXAGGcagaacgua
uacuauucug-3′
(SEQ ID NO: 137)
5′-GalNAc-CXGXXCAGCCXCGCACAGCCAGCACGCACCXGAAXAGG-
3′;
(SEQ ID NO: 137)
5′-FA-CXGXXCAGCCXCGCACAGCCAGCACGCACCXGAAXAGG-3′;
(SEQ ID NO: 137)
5′-DCL-CXGXXCAGCCXCGCACAGCCAGCACGCACCXGAAXAGG-3′;
(SEQ ID NO: 137)
5′-UAMC1110-CXGXXCAGCCXCGCACAGCCAGCACGCACCXGAAXAG
G-3′;
4WJ-D with floxuridine only and derivatives:
(SEQ ID NO: 142)
5′-CCXAXXCAGGXGCGXGCXGGGCXGCAGGXGGCXXXACCXAA-3′;
(targeting aptamer sequences in lower case);
(SEQ ID NO: 143)
5′-CCXAXXCAGGXGCGXGCXGGGCXGCAGGXGGCXXXACCXAAgccuua
guaacgugcuuugaugucgauucgacaggaggc-3′
(targeting aptamer sequences in lower case);
(SEQ ID NO: 144)
5′-CCXAXXCAGGXGCGXGCXGGGCXGCAGGXGGCXXXACCXAAgggacc
gaaaaagaccugacuucuauacuaagucuacguuuccc-3′
(targeting aptamer sequences in lower case);
(SEQ ID NO: 145)
5′-CCXAXXCAGGXGCGXGCXGGGCXGCAGGXGGCXXXACCXAAgcgacu
gguuacccggucg-3′
(targeting aptamer sequences in lower case);
(SEQ ID NO: 146)
5′-CCXAXXCAGGXGCGXGCXGGGCXGCAGGXGGCXXXACCXAAcagaac
guauacuauucug-3′
(SEQ ID NO: 142)
5′-GalNAc-CCXAXXCAGGXGCGXGCXGGGCXGCAGGXGGCXXXACCXA
A-3′;
(SEQ ID NO: 142)
5′-FA-CCXAXXCAGGXGCGXGCXGGGCXGCAGGXGGCXXXACCXAA-
3′;
(SEQ ID NO: 142)
5′-DCL-CCXAXXCAGGXGCGXGCXGGGCXGCAGGXGGCXXXACCXAA-
3′;
(SEQ ID NO: 142)
5′-UAMC1110-CCXAXXCAGGXGCGXGCXGGGCXGCAGGXGGCXXXACC
XAA-3′;
6WJ-A with floxuridine only and derivatives:
(SEQ ID NO: 147)
5′-GAGXAXAXGXXAGGCCXGGGXGAGXCCXXGCGXCXXCXACCG-3′;
(targeting aptamer sequences in lower case);
(SEQ ID NO: 148)
5′-GAGXAXAXGXXAGGCCXGGGXGAGXCCXXGCGXCXXCXACCGgccuu
aguaacgugcuuugaugucgauucgacaggaggc-3′
(targeting aptamer sequences in lower case);
(SEQ ID NO: 149)
5′-GAGXAXAXGXXAGGCCXGGGXGAGXCCXXGCGXCXXCXACCGgggac
cgaaaaagaccugacuucuauacuaagucuacguuuccc-3′
(targeting aptamer sequences in lower case);
(SEQ ID NO: 150)
5′-GAGXAXAXGXXAGGCCXGGGXGAGXCCXXGCGXCXXCXACCGgcgac
ugguuacccggucg-3′
(targeting aptamer sequences in lower case);
(SEQ ID NO: 151)
5′-GAGXAXAXGXXAGGCCXGGGXGAGXCCXXGCGXCXXCXACCGcagaa
cguauacuauucug-3′
(SEQ ID NO: 147)
5′-GalNAc-GAGXAXAXGXXAGGCCXGGGXGAGXCCXXGCGXCXXCXAC
CG-3′;
(SEQ ID NO: 147)
5′-FA-GAGXAXAXGXXAGGCCXGGGXGAGXCCXXGCGXCXXCXACCG-
3′;
(SEQ ID NO: 147)
5′-DCL-GAGXAXAXGXXAGGCCXGGGXGAGXCCXXGCGXCXXCXACCG-
3′;
(SEQ ID NO: 147)
5′-UAMC1110-GAGXAXAXGXXAGGCCXGGGXGAGXCCXXGCGXCXXCX
ACCG-3′;
6WJ-B with floxuridine only and derivatives:
(SEQ ID NO: 152)
5′-CGGXAGAAGACGCAAGGACXXGCXAGXXGXGGXACXGXXCCC-3′;
(targeting aptamer sequences in lower case);
(SEQ ID NO: 153)
5′-CGGXAGAAGACGCAAGGACXXGCXAGXXGXGGXACXGXXCCCgccuu
aguaacgugcuuugaugucgauucgacaggaggc-3′
(targeting aptamer sequences in lower case);
(SEQ ID NO: 154)
5′-CGGXAGAAGACGCAAGGACXXGCXAGXXGXGGXACXGXXCCCgggac
cgaaaaagaccugacuucuauacuaagucuacguuuccc-3′
(targeting aptamer sequences in lower case);
(SEQ ID NO: 155)
5′-CGGXAGAAGACGCAAGGACXXGCXAGXXGXGGXACXGXXCCCgcgac
ugguuacccggucg-3′
(targeting aptamer sequences in lower case);
(SEQ ID NO: 156)
5′-CGGXAGAAGACGCAAGGACXXGCXAGXXGXGGXACXGXXCCCcagaa
cguauacuauucug-3′
(SEQ ID NO: 152)
5′-GalNAc-CGGXAGAAGACGCAAGGACXXGCXAGXXGXGGXACXGXXC
CC-3′;
(SEQ ID NO: 152)
5′-FA-CGGXAGAAGACGCAAGGACXXGCXAGXXGXGGXACXGXXCCC-
3′;
(SEQ ID NO: 152)
5′-DCL-CGGXAGAAGACGCAAGGACXXGCXAGXXGXGGXACXGXXCCC-
3′;
(SEQ ID NO: 152)
5′-UAMC1110-CGGXAGAAGACGCAAGGACXXGCXAGXXGXGGXACXGX
XCCC-3′;
6WJ-C with floxuridine only and derivatives:
(SEQ ID NO: 157)
5′-GGGAACAGXACCACAACXAGXGXCCCGGGAXAGGGACAXACA-3′;
(targeting aptamer sequences in lower case);
(SEQ ID NO: 158)
5′-GGGAACAGXACCACAACXAGXGXCCCGGGAXAGGGACAXACAgccuu
aguaacgugcuuugaugucgauucgacaggaggc-3′
(targeting aptamer sequences in lower case);
(SEQ ID NO: 159)
5′-GGGAACAGXACCACAACXAGXGXCCCGGGAXAGGGACAXACAgggac
cgaaaaagaccugacuucuauacuaagucuacguuuccc-3′
(targeting aptamer sequences in lower case);
(SEQ ID NO: 160)
5′-GGGAACAGXACCACAACXAGXGXCCCGGGAXAGGGACAXACAgcgac
ugguuacccggucg-3′
(targeting aptamer sequences in lower case);
(SEQ ID NO: 161)
5′-GGGAACAGXACCACAACXAGXGXCCCGGGAXAGGGACAXACAcagaa
cguauacuauucug-3′
(SEQ ID NO: 157)
5′-GalINAc-GGGAACAGXACCACAACXAGXGXCCCGGGAXAGGGACAX
ACA-3′;
(SEQ ID NO: 157)
5′-FA-GGGAACAGXACCACAACXAGXGXCCCGGGAXAGGGACAXACA-
3′;
(SEQ ID NO: 157)
5′-DCL-GGGAACAGXACCACAACXAGXGXCCCGGGAXAGGGACAXACA-
3′;
(SEQ ID NO: 157)
5′-UAMC1110-GGGAACAGXACCACAACXAGXGXCCCGGGAXAGGGACA
XACA-3′;
6WJ-D with floxuridine only and derivatives:
(SEQ ID NO: 162);
5′-XGXAXGXCCCXAXCCCGGGAXGCCCAGGCCXAACAXAXACXC-3′
(targeting aptamer sequences in lower case)
(SEQ ID NO: 163)
5′-XGXAXGXCCCXAXCCCGGGAXGCCCAGGCCXAACAXAXACXCgccuu
aguaacgugcuuugaugucgauucgacaggaggc-3′
(targeting aptamer sequences in lower case);
(SEQ ID NO: 164)
5′-XGXAXGXCCCXAXCCCGGGAXGCCCAGGCCXAACAXAXACXCgggac
cgaaaaagaccugacuucuauacuaagucuacguuuccc-3′
(targeting aptamer sequences in lower case);
(SEQ ID NO: 165)
5′-XGXAXGXCCCXAXCCCGGGAXGCCCAGGCCXAACAXAXACXCgcgac
ugguuacccggucg-3′
(targeting aptamer sequences in lower case);
(SEQ ID NO: 166)
5′-XGXAXGXCCCXAXCCCGGGAXGCCCAGGCCXAACAXAXACXCcagaa
cquauacuauucug-3′
(SEQ ID NO: 162)
5′-GalNAc-XGXAXGXCCCXAXCCCGGGAXGCCCAGGCCXAACAXAXAC
XC-3′;
(SEQ ID NO: 162)
5′-FA-XGXAXGXCCCXAXCCCGGGAXGCCCAGGCCXAACAXAXACXC-
3′;
(SEQ ID NO: 162)
5′-DCL-XGXAXGXCCCXAXCCCGGGAXGCCCAGGCCXAACAXAXACXC-
3′;
or
(SEQ ID NO: 162)
5′-UAMC1110-XGXAXGXCCCXAXCCCGGGAXGCCCAGGCCXAACAXAX
ACXC-3′, wherein X is floxuridine(5-fluorodeoxy-
uridine, UB5F).
In some embodiments, the disclosed RNA
oligonucleotide has one of the following
unmodified sequences:
3WJ-A:
(SEQ ID NO: 167)
5′-UUGCCAUGUGUAUGUGGG-3′;
(SEQ ID NO: 168)
5′-UUGCCAUGUGUAUGUGGGGGCCUUAGUAACGUGCUUUGAUGUCGAUU
CGACAGGAGGC-3′;
(SEQ ID NO: 169)
5′-UUGCCAUGUGUAUGUGGGGGGACCGAAAAAGACCUGACUUCUAUACU
AAGUCUACGUUUCCC-3′;
(SEQ ID NO: 170)
5′-UUGCCUGUGUAUGUGGGGCGACUGGUUACCCGGUCG-3′;
(SEQ ID NO: 171)
5′-UUGCCAUGUGUAUGUGGGCAGAACGUAUACUAUUCUG-3′;
(SEQ ID NO: 167)
5′-GalNAc-UUGCCAUGUGUAUGUGGG-3′;
(SEQ ID NO: 167)
5′-FA-UUGCCAUGUGUAUGUGGG-3′;
(SEQ ID NO: 167)
5′-DCL-UUGCCAUGUGUAUGUGGG-3′;
(SEQ ID NO: 167)
5′-UAMC1110-UUGCCAUGUGUAUGUGGG-3′;
3WJ-B:
(SEQ ID NO: 172)
5′-CCCACAUACUUUGUUGAUCC-3′;
(SEQ ID NO: 173)
5′-CCCACAUACUUUGUUGAUCCGCCUUAGUAACGUGCUUUGAUGUCGAU
UCGACAGGAGGC-3′;
(SEQ ID NO: 174)
5′-CCCACAUACUUUGUUGAUCCGGGACCGAAAAAGACCUGACUUCUAUA
CUAAGUCUACGUUUCCC-3′;
(SEQ ID NO: 175)
5′-CCCACAUACUUUGUUGAUCCGCGACUGGUUACCCGGUCG-3′;
(SEQ ID NO: 176)
5′-CCCACAUACUUUGUUGAUCCCAGAACGUAUACUAUUCUG-3′;
(SEQ ID NO: 173)
5′-GalNAc-CCCACAUACUUUGUUGAUCC-3′;
(SEQ ID NO: 173)
5′-FA-CCCACAUACUUUGUUGAUCC-3′;
(SEQ ID NO: 173)
5′-DCL-CCCACAUACUUUGUUGAUCC-3′;
(SEQ ID NO: 173)
5′-UAMC1110-CCCACAUACUUUGUUGAUCC-3′;
3WJ-C:
(SEQ ID NO: 177)
5′-GGAUCAAUCAUGGCAA-3′;
(SEQ ID NO: 178)
5′-GGAUCAAUCAUGGCAAGCCUUAGUAACGUGCUUUGAUGUCGAUUCGA
CAGGAGGC-3′;
(SEQ ID NO: 179)
5′-GGAUCAAUCAUGGCAAGGGACCGAAAAAGACCUGACUUCUAUACUAA
GUCUACGUUUCCC-3′;
(SEQ ID NO: 180)
5′-GGAUCAAUCAUGGCAAGCGACUGGUUACCCGGUCG-3′;
(SEQ ID NO: 181)
5′-GGAUCAAUCAUGGCAACAGAACGUAUACUAUUCUG-3′;
(SEQ ID NO: 177)
5′-GalNAc-GGAUCAAUCAUGGCAA-3′;
(SEQ ID NO: 177)
5′-FA-GGAUCAAUCAUGGCAA-3′;
(SEQ ID NO: 177)
5′-DCL-GGAUCAAUCAUGGCAA-3′;
(SEQ ID NO: 177)
5′-UAMC1110-GGAUCAAUCAUGGCAA-3′;
4WJ-A:
(SEQ ID NO: 182)
5′-UUAGGUAAAGCCACCUGCAGGUGCUACCGAUGUAAUUCAA-3′;
(SEQ ID NO: 183)
5′-UUAGGUAAAGCCACCUGCAGGUGCUACCGAUGUAAUUCAAGCCUUAG
UAACGUGCUUUGAUGUCGAUUCGACAGGAGGC-3′;
(SEQ ID NO: 184)
5′-UUAGGUAAAGCCACCUGCAGGUGCUACCGAUGUAAUUCAAGGGACCG
AAAAAGACCUGACUUCUAUACUAAGUCUACGUUUCCC-3′;
(SEQ ID NO: 185)
5′-UUAGGUAAAGCCACCUGCAGGUGCUACCGAUGUAAUUCAAGCGACUG
GUUACCCGGUCG-3′;
(SEQ ID NO: 186)
5′-UUAGGUAAAGCCACCUGCAGGUGCUACCGAUGUAAUUCAACAGAACG
UAUACUAUUCUG-3′;
(SEQ ID NO: 182)
5′-GalNAc-UUAGGUAAAGCCACCUGCAGGUGCUACCGAUGUAAUUCA
A-3′;
(SEQ ID NO: 182)
5′-FA-UUAGGUAAAGCCACCUGCAGGUGCUACCGAUGUAAUUCAA-3′;
(SEQ ID NO: 182)
5′-DCL-UUAGGUAAAGCCACCUGCAGGUGCUACCGAUGUAAUUCAA-
3′;
(SEQ ID NO: 182)
5′-UAMC1110UUAGGUAAAGCCACCUGCAGGUGCUACCGAUGUAAUUCA
A-3′;
4WJ-B:
(SEQ ID NO: 187)
5′-UUGAAUUACAUCGGUAGCACGGGCUGUGCGAGGCUGAACAG-3′;
(SEQ ID NO: 188)
5′-UUGAAUUACAUCGGUAGCACGGGCUGUGCGAGGCUGAACAGGCCUUA
GUAACGUGCUUUGAUGUCGAUUCGACAGGAGGC-3′;
(SEQ ID NO: 189)
5′-UUGAAUUACAUCGGUAGCACGGGCUGUGCGAGGCUGAACAGGGGACC
GAAAAAGACCUGACUUCUAUACUAAGUCUACGUUUCCC-3′;
(SEQ ID NO: 190)
5′-UUGAAUUACAUCGGUAGCACGGGCUGUGCGAGGCUGAACAGGCGACU
GGUUACCCGGUCG-3′;
(SEQ ID NO: 191)
5′-UUGAAUUACAUCGGUAGCACGGGCUGUGCGAGGCUGAACAGCAGAAC
GUAUACUAUUCUG-3′;
(SEQ ID NO: 187)
5′-GalNAc-UUGAAUUACAUCGGUAGCACGGGCUGUGCGAGGCUGAACA
G-3′;
(SEQ ID NO: 187)
5′-FA-UUGAAUUACAUCGGUAGCACGGGCUGUGCGAGGCUGAACAG-
3′;
(SEQ ID NO: 187)
5′-DCL-UUGAAUUACAUCGGUAGCACGGGCUGUGCGAGGCUGAACAG-
3′;
(SEQ ID NO: 187)
5′-UAMC1110-UUGAAUUACAUCGGUAGCACGGGCUGUGCGAGGCUGAA
CAG-3′;
4WJ-C:
(SEQ ID NO: 192)
5′-CUGUUCAGCCUCGCACAGCCAGCACGCACCUGAAUAGG-3′;
(SEQ ID NO: 193)
5′-CUGUUCAGCCUCGCACAGCCAGCACGCACCUGAAUAGGGCCUUAGUA
ACGUGCUUUGAUGUCGAUUCGACAGGAGGC-3′;
(SEQ ID NO: 194)
5′-CUGUUCAGCCUCGCACAGCCAGCACGCACCUGAAUAGGGGGACCGAA
AAAGACCUGACUUCUAUACUAAGUCUACGUUUCCC-3′;
(SEQ ID NO: 195)
5′-CUGUUCAGCCUCGCACAGCCAGCACGCACCUGAAUAGGGCGACUGGU
UACCCGGUCG-3′;
(SEQ ID NO: 196)
5′-CUGUUCAGCCUCGCACAGCCAGCACGCACCUGAAUAGGCAGAACGUA
UACUAUUCUG-3′;
(SEQ ID NO: 192)
5′-GalNAc-CUGUUCAGCCUCGCACAGCCAGCACGCACCUGAAUAGG-
3′;
(SEQ ID NO: 192)
5′-FA-CUGUUCAGCCUCGCACAGCCAGCACGCACCUGAAUAGG-3′;
(SEQ ID NO: 192)
5′-DCL-CUGUUCAGCCUCGCACAGCCAGCACGCACCUGAAUAGG-3′;
(SEQ ID NO: 192)
5′-UAMC1110-CUGUUCAGCCUCGCACAGCCAGCACGCACCUGAAUAG
G-3′;
4WJ-D:
(SEQ ID NO: 197)
5′-CCUAUUCAGGUGCGUGCUGGGCUGCAGGUGGCUUUACCUAA-3′;
(SEQ ID NO: 198)
5′-CCUAUUCAGGUGCGUGCUGGGCUGCAGGUGGCUUUACCUAAGCCUUA
GUAACGUGCUUUGAUGUCGAUUCGACAGGAGGC-3′;
(SEQ ID NO: 199)
5′-CCUAUUCAGGUGCGUGCUGGGCUGCAGGUGGCUUUACCUAAGGGACC
GAAAAAGACCUGACUUCUAUACUAAGUCUACGUUUCCC-3′;
(SEQ ID NO: 200)
5′-CCUAUUCAGGUGCGUGCUGGGCUGCAGGUGGCUUUACCUAAGCGACU
GGUUACCCGGUCG-3′;
(SEQ ID NO: 201)
5′-CCUAUUCAGGUGCGUGCUGGGCUGCAGGUGGCUUUACCUAACAGAAC
GUAUACUAUUCUG-3′;
(SEQ ID NO: 197);
5′-GalNAc-CCUAUUCAGGUGCGUGCUGGGCUGCAGGUGGCUUUACCUA
A-3′
(SEQ ID NO: 197)
5′-FA-CCUAUUCAGGUGCGUGCUGGGCUGCAGGUGGCUUUACCUAA-
3′;
(SEQ ID NO: 197)
5′-DCL-CCUAUUCAGGUGCGUGCUGGGCUGCAGGUGGCUUUACCUAA-
3′;
(SEQ ID NO: 197)
5′-UAMC1110-CCUAUUCAGGUGCGUGCUGGGCUGCAGGUGGCUUUACC
UAA-3′;
6WJ-A:
(SEQ ID NO: 202)
5′-GAGUAUAUGUUAGGCCUGGGUGAGUCCUUGCGUCUUCUACCG-3′;
(SEQ ID NO: 203)
5′-GAGUAUAUGUUAGGCCUGGGUGAGUCCUUGCGUCUUCUACCGGCCUU
AGUAACGUGCUUUGAUGUCGAUUCGACAGGAGGC-3′;
(SEQ ID NO: 204)
5′-GAGUAUAUGUUAGGCCUGGGUGAGUCCUUGCGUCUUCUACCGGGGAC
CGAAAAAGACCUGACUUCUAUACUAAGUCUACGUUUCCC-3′;
(SEQ ID NO: 205)
5′-GAGUAUAUGUUAGGCCUGGGUGAGUCCUUGCGUCUUCUACCGGCGAC
UGGUUACCCGGUCG-3′;
(SEQ ID NO: 206)
5′-GAGUAUAUGUUAGGCCUGGGUGAGUCCUUGCGUCUUCUACCGCAGAA
CGUAUACUAUUCUG-3′;
(SEQ ID NO: 202)
5′-GalNAc-GAGUAUAUGUUAGGCCUGGGUGAGUCCUUGCGUCUUCUAC
CG-3′;
(SEQ ID NO: 202)
5′-FA-GAGUAUAUGUUAGGCCUGGGUGAGUCCUUGCGUCUUCUACCG-
3′;
(SEQ ID NO: 202)
5′-DCL-GAGUAUAUGUUAGGCCUGGGUGAGUCCUUGCGUCUUCUACCG-
3′;
(SEQ ID NO: 202)
5′-UAMC1110-GAGUAUAUGUUAGGCCUGGGUGAGUCCUUGCGUCUUCU
ACCG-3′;
6WJ-B:
(SEQ ID NO: 207)
5′-CGGUAGAAGACGCAAGGACUUGCUAGUUGUGGUACUGUUCCC-3′;
(SEQ ID NO: 208)
5′-CGGUAGAAGACGCAAGGACUUGCUAGUUGUGGUACUGUUCCCGCCUU
AGUAACGUGCUUUGAUGUCGAUUCGACAGGAGGC-3′;
(SEQ ID NO: 209)
5′-CGGUAGAAGACGCAAGGACUUGCUAGUUGUGGUACUGUUCCCGGGAC
CGAAAAAGACCUGACUUCUAUACUAAGUCUACGUUUCCC-3′;
(SEQ ID NO: 210)
5′-CGGUAGAAGACGCAAGGACUUGCUAGUUGUGGUACUGUUCCCGCGAC
UGGUUACCCGGUCG-3′;
(SEQ ID NO: 211)
5′-CGGUAGAAGACGCAAGGACUUGCUAGUUGUGGUACUGUUCCCCAGAA
CGUAUACUAUUCUG-3′;
(SEQ ID NO: 207)
5′-GalNAc-CGGUAGAAGACGCAAGGACUUGCUAGUUGUGGUACUGUUC
CC-3′;
(SEQ ID NO: 207)
5′-FA CGGUAGAAGACGCAAGGACUUGCUAGUUGUGGUACUGUUCCC-
3′;
(SEQ ID NO: 207)
5′-DCL CGGUAGAAGACGCAAGGACUUGCUAGUUGUGGUACUGUUCCC-
3′;
(SEQ ID NO: 207)
5′-UAMC1110-CGGUAGAAGACGCAAGGACUUGCUAGUUGUGGUACUGU
UCCC-3′;
6WJ-C:
(SEQ ID NO: 212)
5′-GGGAACAGUACCACAACUAGUGUCCCGGGAUAGGGACAUACA-3′;
(SEQ ID NO: 213)
5′-GGGAACAGUACCACAACUAGUGUCCCGGGAUAGGGACAUACAGCCUU
AGUAACGUGCUUUGAUGUCGAUUCGACAGGAGGC-3′;
(SEQ ID NO: 214)
5′-GGGAACAGUACCACAACUAGUGUCCCGGGAUAGGGACAUACAGGGAC
CGAAAAAGACCUGACUUCUAUACUAAGUCUACGUUUCCC-3′;
(SEQ ID NO: 215)
5′-GGGAACAGUACCACAACUAGUGUCCCGGGAUAGGGACAUACAGCGAC
UGGUUACCCGGUCG-3′;
(SEQ ID NO: 216)
5′-GGGAACAGUACCACAACUAGUGUCCCGGGAUAGGGACAUACACAGAA
CGUAUACUAUUCUG 3′;
(SEQ ID NO: 212)
5′-GalNAc-GGGAACAGUACCACAACUAGUGUCCCGGGAUAGGGACAUA
CA-3′;
(SEQ ID NO: 212)
5′-FA GGGAACAGUACCACAACUAGUGUCCCGGGAUAGGGACAUACA-
3′;
(SEQ ID NO: 212)
5′-DCL-GGGAACAGUACCACAACUAGUGUCCCGGGAUAGGGACAUACA-
3′;
(SEQ ID NO: 212)
5′-UAMC1110-GGGAACAGUACCACAACUAGUGUCCCGGGAUAGGGACA
UACA-3′;
6WJ-D:
(SEQ ID NO: 217)
5′-UGUAUGUCCCUAUCCCGGGAUGCCCAGGCCUAACAUAUACUC-3′;
(SEQ ID NO: 218)
5′-UGUAUGUCCCUAUCCCGGGAUGCCCAGGCCUAACAUAUACUCGCCUU
AGUAACGUGCUUUGAUGUCGAUUCGACAGGAGGC-3′;
(SEQ ID NO: 219)
5′-UGUAUGUCCCUAUCCCGGGAUGCCCAGGCCUAACAUAUACUCGGGAC
CGAAAAAGACCUGACUUCUAUACUAAGUCUACGUUUCCC-3′;
(SEQ ID NO: 220)
5′-UGUAUGUCCCUAUCCCGGGAUGCCCAGGCCUAACAUAUACUCGCGAC
UGGUUACCCGGUCG-3′;
(SEQ ID NO: 221)
5′-UGUAUGUCCCUAUCCCGGGAUGCCCAGGCCUAACAUAUACUCCAGAA
CGUAUACUAUUCUG-3′;
(SEQ ID NO: 217)
5′-GalNAc-UGUAUGUCCCUAUCCCGGGAUGCCCAGGCCUAACAUAUAC
UC-3′;
(SEQ ID NO: 217)
5′-FA-UGUAUGUCCCUAUCCCGGGAUGCCCAGGCCUAACAUAUACUC-
3′;
(SEQ ID NO: 217)
5′-DCL-UGUAUGUCCCUAUCCCGGGAUGCCCAGGCCUAACAUAUACUC-
3′;
or
(SEQ ID NO: 217)
5′-UAMC1110-UGUAUGUCCCUAUCCCGGGAUGCCCAGGCCUAACAUAU
ACUC-3′.
(SEQ ID NO: 222)
5′ XXGYYAXGXGXAXGXGGG Cholesterol-3′;
(SEQ ID NO: 223)
5′ YYYAYAXAYXXXGXXGAXYY Cholesterol-3′;
(SEQ ID NO: 224)
5′ GGAXYAAXYAXGGYAA-Cholesterol-3′;
(SEQ ID NO: 225)
5′ XXAGGXAAAGYYAYYXGYAGGXGYXAYYGAXGXAAXXYAA-
Cholesterol-3′;
(SEQ ID NO: 226)
5′ XXGAAXXAYAXYGGXAGYAYGGGYXGXGYGAGGYXGAAYAG-
Cholesterol-3′;
(SEQ ID NO: 227)
5′ YXGXXYAGYYXYGYAYAGYYAGYAYGYAYYXGAAXAGG-
Cholesterol-3′;
(SEQ ID NO: 228)
5′ YYXAXXYAGGXGYGXGYXGGGYXGYAGGXGGYXXXAYYXAA-
Cholesterol-3′;
(SEQ ID NO: 229)
5′ GAGXAXAXGXXAGGYYXGGGXGAGXYYXXGYGXYXXYXAYYG-
Cholesterol-3′;
(SEQ ID NO: 230)
5′ YGGXAGAAGAYGYAAGGAYXXGYXAGXXGXGGXAYXGXXYYY-
Cholesterol-3′;
(SEQ ID NO: 231)
5′ GGGAAYAGXAYYAYAAYXAGXGXYYYGGGAXAGGGAYAXAYA-
Cholesterol-3′;
(SEQ ID NO: 232)
5′ XGXAXGXYYYXAXYYYGGGAXGYYYAGGYYXAAYAXAXAYXY-
Cholesterol-3′, wherein X is floxuridine(5-
fluorodeoxyuridine, UB5F), and wherein Y is
gemcitabine(2′, 2′-difluoro-2′-deoxycytidine,.
dFdC, CR2FF)
In some embodiments, the disclosed RNA
oligonucleotide has one of the following
sequences:
(SEQ ID NO: 233)
5′ UUGYYAUGUGUAUGUGGG Cholesterol-3′;
(SEQ ID NO: 234)
5′ YYYAYAUAYUUUGUUGAUYY Cholesterol-3′;
(SEQ ID NO: 235)
5′ GGAUYAAUYAUGGYAA Cholesterol-3′;
(SEQ ID NO: 236)
5′ UUAGGUAAAGYYAYYUGYAGGUGYUAYYGAUGUAAUUYAA
Cholesterol-3′;
(SEQ ID NO: 237)
5′ UUGAAUUAYAUYGGUAGYAYGGGYUGUGYGAGGYUGAAYAG
Cholesterol-3′;
(SEQ ID NO: 238)
5′ YUGUUYAGYYUYGYAYAGYYAGYAYGYAYYUGAAUAGG
Cholesterol-3′;
(SEQ ID NO: 239)
5′ YYUAUUYAGGUGYGUGYUGGGYUGYAGGUGGYUUUAYYUAA
Cholesterol-3′;
(SEQ ID NO: 240)
5′ GAGUAUAUGUUAGGYYUGGGUGAGUYYUUGYGUYUUYUAYYG
Cholesterol-3′;
(SEQ ID NO: 241)
5′ YGGUAGAAGAYGYAAGGAYUUGYUAGUUGUGGUAYUGUUYYY
Cholesterol-3′;
(SEQ ID NO: 242)
5′ GGGAAYAGUAYYAYAAYUAGUGUYYYGGGAUAGGGAYAUAYA
Cholesterol-3′;
(SEQ ID NO: 243)
5′ UGUAUGUYYYUAUYYYGGGAUGYYYAGGYYUAAYAUAUAYUY
Cholesterol-3′, wherein Y is gemcitabine(2′,
2′-difluoro-2′- deoxycytidine, dFdC, CR2FF).
(SEQ ID NO: 244)
5′ XXGCCAXGXGXAXGXGGG-Cholesterol-3′;
(SEQ ID NO: 245)
5′ CCCACAXACXXXGXXGAXCC-Cholesterol-3′;
(SEQ ID NO: 246)
5′ GGAXCAAXCAXGGCAA-Cholesterol-3′;
(SEQ ID NO: 247)
5′ XXAGGXAAAGCCACCXGCAGGXGCXACCGAXGXAAXXCAA-
Cholesterol-3′;
(SEQ ID NO: 248)
5′ XXGAAXXACAXCGGXAGCACGGGCXGXGCGAGGCXGAACAG-
Cholesterol-3′;
(SEQ ID NO: 249)
5′ CXGXXCAGCCXCGCACAGCCAGCACGCACCXGAAXAGG-
Cholesterol-3′;
(SEQ ID NO: 250)
5′ CCXAXXCAGGXGCGXGCXGGGCXGCAGGXGGCXXXACCXAA-
Cholesterol-3′;
(SEQ ID NO: 251)
5′ GAGXAXAXGXXAGGCCXGGGXGAGXCCXXGCGXCXXCXACCG-
Cholesterol-3′;
(SEQ ID NO: 252)
5′ CGGXAGAAGACGCAAGGACXXGCXAGXXGXGGXACXGXXCCC-
Cholesterol-3′;
(SEQ ID NO: 253)
5′ GGGAACAGXACCACAACXAGXGXCCCGGGAXAGGGACAXACA-
Cholesterol-3′;
(SEQ ID NO: 254)
5′ XGXAXGXCCCXAXCCCGGGAXGCCCAGGCCXAACAXAXACXC-
Cholesterol-3′, wherein X is floxuridine(5-
fluorodeoxyuridine, UB5F).
(SEQ ID NO: 255)
5′ UUGCCAUGUGUAUGUGGG-Cholesterol-3′;
(SEQ ID NO: 256)
5′ CCCACAUACUUUGUUGAUCC-Cholesterol-3′;
(SEQ ID NO: 257)
5′ GGAUCAAUCAUGGCAA-Cholesterol-3′;
(SEQ ID NO: 258)
5′ UUAGGUAAAGCCACCUGCAGGUGCUACCGAUGUAAUUCAA-
Cholesterol-3′;
(SEQ ID NO: 259)
5′ UUGAAUUACAUCGGUAGCACGGGCUGUGCGAGGCUGAACAG-
Cholesterol-3′;
(SEQ ID NO: 260)
5′ CUGUUCAGCCUCGCACAGCCAGCACGCACCUGAAUAGG-
Cholesterol-3′;
(SEQ ID NO: 261)
5′ CCUAUUCAGGUGCGUGCUGGGCUGCAGGUGGCUUUACCUAA-
Cholesterol-3′;
(SEQ ID NO: 262)
5′ GAGUAUAUGUUAGGCCUGGGUGAGUCCUUGCGUCUUCUACCG-
Cholesterol-3′;
(SEQ ID NO: 263)
5′ CGGUAGAAGACGCAAGGACUUGCUAGUUGUGGUACUGUUCCC-
Cholesterol-3′;
(SEQ ID NO: 264)
5′ GGGAACAGUACCACAACUAGUGUCCCGGGAUAGGGACAUACA-
Cholesterol-3′;
(SEQ ID NO: 265)
5′ UGUAUGUCCCUAUCCCGGGAUGCCCAGGCCUAACAUAUACUC-
Cholesterol-3′;
Survivin-Sense with extension
(SEQ ID NO: 266)
5′ XXGYAGGXXYYXXAXYXGXYAXXAYAXAYAYYYX-Cholesterol-
3′;
(SEQ ID NO: 267)
5′ UUGYAGGUUYYUUAUYUGUYAUUAYAUAYAYYYU-Cholesterol-
3′;
(SEQ ID NO: 268)
5′ XXGCAGGXXCCXXAXCXGXCAXXACAXACACCCX-Cholesterol-
3′;
(SEQ ID NO: 269)
5′ UUGCAGGUUCCUUAUCUGUCAUUACAUACACCCU-Cholesterol-
3′.
RRM2-Sense with extension
(SEQ ID NO: 270)
5′ XXGYGAXXXAGYYAAGAAGXXYAXXAYAXAYAYYYX-
Cholesterol-3′;
(SEQ ID NO: 271)
5′ UUGYGAUUUAGYYAAGAAGUUYAUUAYAUAYAYYYU-
Cholesterol-3′;
(SEQ ID NO: 272)
5′ XXGCGAXXXAGCCAAGAAGXXCAXXACAXACACCCX-
Cholesterol-3′;
(SEQ ID NO: 273)
5′ UUGCGAUUUAGCCAAGAAGUUCAUUACAUACACCCU-
Cholesterol-3′;
wherein X is floxuridine(5-fluorodeoxyuridine,
UB5F), wherein Y is gemcitabine (2′, 2′-
difluoro-2′-deoxycytidine, dFdC, CR2FF),
wherein C, U are 2F′ modified C, U, wherein A,
G are unmodified.
Survivin-Anti Sense
(SEQ ID NO: 1)
5′ UGACAGAUAAGGAACCUGC 3′.
RRM2-Anti Sense
(SEQ ID NO: 3)
5′ UGAACUUCUUGGCUAAAUCGC 3′.
Survivin-Anti Sense-Extension
(SEQ ID NO: 4)
5′ GGGUGUAUGUAAUGACAGAUAAGGAACCUGC 3′.
RRM2-Anti Sense
(SEQ ID NO: 2)
5′ GGGUGUAUGUAAUGAACUUCUUGGCUAAAUCGC 3′;
Extension
(SEQ ID NO: 5)
5′ GGGUGUAUGUAA 3′, wherein A, C, G, U are
unmodified.

The modular RNA nanostructures can be composed of 3, 4, 5, 6, 7, 8, 9 or more synthetic single-stranded RNA oligonucleotides that can self-assemble into the RNA nanostructures via hybridization. Each synthetic single-stranded RNA oligonucleotide can be about 16 to about 120 bases in length. The exact sequence of each synthetic single-stranded RNA oligonucleotide in each modular RNA nanostructures can be designed such that they achieve specific physical characteristics when assembled with 2 or more other synthetic single-stranded RNA oligonucleotides. The RNA nanostructures can be composed of 3, 4, 5, 6, 7, 8, 9, or more synthetic RNA oligonucleotides. The synthetic RNA oligonucleotides can be designed such that they form highly ordered 2-D and/or 3-D structures upon self-assembly. The RNA nanostructures can have 3, 4, 5, 6, 7, 8, 9, or more double-stranded arms (DAs) that stem off of a core domain. The DAs can be symmetrically or asymmetrically arranged around the core domain.

The core domain can have 0-4 symmetric or asymmetric bulge nucleotides separating individual DAs, which can allow for optimization of thermodynamic stability, steric constraints, and/or structural arrangements of individual loops. Changes in duplex sequence (the DAs) and number unpaired core nucleotides can both affect thermodynamic stability of the RNA nanostructures. Thus the physical properties and functional characteristics can be optimized by altering the sequence of the synthetic RNA oligonucleotides that form the RNA nanostructures.

The melting temperature (Tm) of the RNA nanostructure can be about 65° C. or more. In some aspects, the melting temperature of the RNA nanostructure can be greater than 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. In some aspects, the Tm of the RNA nanostructure can be 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.

The synthetic RNA oligonucleotides can be single-stranded. Each individual synthetic RNA oligonucleotide can be composed of 16-120 nucleotides. The nucleotides can be native ribonucleotide or can be modified. In some aspects, the synthetic RNA oligonucleotide(s) can be 2′ modified. The 2′ or other modification can be a 2′Fluoro-, 2′O-methyl-, LNA- or any other backbone, sugar, or base modified ribonucleotide or any combination of native, backbone, sugar, and base modified ribonucleotides.

Modifications are further discussed elsewhere herein. Each synthetic RNA oligonucleotide can be composed of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 nucleotides or any range therein. Each synthetic RNA oligonucleotide can be designed and configured such that it can self assemble with 2, 3, 4, 5, 6, 7, 8, or more other synthetic RNA oligonucleotides into a RNA nanostructure as described herein.

In addition to the nucleoside analogues described herein, the remaining nucleotides can be unmodified or modified nucleotides. The modifications can be 5′-terminal modifications and/or 3′-terminal modifications and/or 2′-internal sugar modifications and/or base-internal modifications. Typical 5′ terminal modifications include amino, carboxy, phosphate, thiol, maleimide, alkyne, cholesterol, aldehyde, carbon spacers, Peg-spacer, doubler, trebler, photocleavable amino, photocleavable spacer, fluorophores (e.g. Cyanine 3, 3.5, 5, 5.5, 7, Fluorescein, etc.), biotin, desthiobiotin, digoxigenin, quenchers (dabcyl, dabsyl, BlackHole, BBQ650, etc.) or other 5′ modifications known to an experienced user of the art. Typical 3′ terminal modifications include amino, carboxy, phosphate, thiol, alkyne, cholesterol, carbon spacers, Peg-spacer, fluorophores (e.g. Cyanine 3, 3.5, 5, 5.5, 7, Fluorescein, etc.), biotin, desthiobiotin, digoxigenin, quenchers (dabcyl, dabsyl, BlackHole, BBQ650, etc.) or other 3′ modifications known to an experienced user of the art. Typical internal modifications include amino-dA, amino-dC, amino-dT, carboxy-dT, 2′O-propargyl, 2′amino, 2′fluoro, 2′methoxy, 5-ethynyl-dU, C8-alkyne-dC, C8-alkyne-dT, carbon spacers, Peg-spacer, fluorophores (e.g. Cyanine 3, 3.5, 5, 5.5, 7, Fluorescein, etc.), biotin, desthiobiotin, digoxigenin, quenchers (dabcyl, dabsyl, BlackHole, BBQ650, etc.) or other 5′ modifications known to an experienced user of the art. The modification can be an alkyne group attached to a nucleotide. The modification can be a functional group attached to a nucleotide. One or more of the terminal (e.g. the 5′ and/or 3′ end) nucleotides can be modified in a synthetic RNA oligonucleotide.

The RNA nanostructure, when measured along its longest or largest dimension, can have a size of up to a micrometer. The size of the RNA nanostructure, when measured along its longest or largest dimension, can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 250, 300, 350, 400, 450, 500, 600, 700, 800 or about 900 nm. In some aspects, the size of the RNA nanostructure, when measured along its longest or largest dimension, can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nm or any range of values therein. In some aspects, the size of the RNA nanostructure, when measured along its longest or largest dimension, can be about 1-30, 1-40, 1-50, 10-50, 10-40, 10-30, 30-50, 30-40, or 40-50 nm. In some aspects, the RNA nanostructure can be substantially spherical. In other aspects, the RNA nanostructure can be trigonal planar, trigonal pyramidal, T-shaped, tetrahedral, square planar, seesaw, trigonal bipyramidal, square pyramidal, pentagonal planar, octahedral, trigonal prismatic, pentagonal pyramidal, pentagonal bipyramidal, square antiprismatic, tricapped trigonal prismatic, capped square antiprismatic, arrowhead shaped, arrow tail shaped, X-shaped, or a distorted version of any of these shapes.

A principle for designing self-assembled nucleic acid nanostructures is that sequence complementarity in nucleic acid strands is encoded such that, by pairing up complementary segments, the nucleic acid strands self-organize into a predefined nanostructure under appropriate physical conditions. From this basic principle (see, e.g., Seeman N.C. J. Theor. Biol. 99: 237, 1982, incorporated by reference herein), researchers have created diverse synthetic nucleic acid nanostructures (see, e.g., Seeman N.C. Nature 421: 427, 2003; Shih W. M. et al. Curr. Opin. Struct. Biol. 20: 276, 2010, each of which is incorporated by reference herein). Examples of nucleic acid (e.g., DNA) nanostructures, and methods of producing such structures, that may be used in accordance with the present disclosure are known and include, without limitation, lattices (see, e.g., Winfree E. et al. Nature 394: 539, 1998; Yan H. et al. Science 301: 1882, 2003; Yan H. et al. Proc. Natl. Acad. of Sci. USA 100; 8103, 2003; Liu D. et al. J. Am. Chem. Soc. 126: 2324, 2004; Rothemund P. W. K. et al. PLoS Biology 2:2041, 2004, each of which is incorporated by reference herein), ribbons (see, e.g., Park S. H. et al. Nano Lett. 5: 729, 2005; Yin P. et al. Science 321: 824, 2008, each of which is incorporated by reference herein), tubes (see, e.g., Yan H. Science, 2003; P. Yin, 2008, each of which is incorporated by reference herein), finite two-dimensional and three dimensional objects with defined shapes (see, e.g., Chen J. et al. Nature 350: 631, 1991; Rothemund P. W. K., Nature, 2006; He Y. et al. Nature 452: 198, 2008; Ke Y. et al. Nano. Lett. 9: 2445, 2009; Douglas S. M. et al. Nature 459: 414, 2009; Dietz H. et al. Science 325: 725, 2009; Andersen E. S. et al. Nature 459: 73, 2009; Liedl T. et al. Nature Nanotech. 5: 520, 2010; Han D. et al. Science 332: 342, 2011, each of which is incorporated by reference herein), and macroscopic crystals (see, e.g., Meng J. P. et al. Nature 461: 74, 2009, incorporated by reference herein). The synthetic RNA oligonucleotides can be single-stranded nucleic acids, double-stranded nucleic acids, or a combination of single-stranded and double-stranded nucleic acids.

The RNA nanostructure components (synthetic RNA oligonucleotides and modular RNA motifs can be designed using a computer aided design methodology described herein. Although the computer design is demonstrated using specific RNA nanostructures, it will be appreciated that the principles taught therein will be able to be extrapolated to any desired RNA nanostructure by the skilled artisan.

Each synthetic RNA oligonucleotide can be designed such that when it is combined with 2 or more additional synthetic RNA oligonucleotides that base pairing occurs to produce a highly ordered 2-D and 3-D structure having 3 or more DAs surrounding a core domain that may or may not contain base-pairing between strands and can include 0-4 nucleotides that form symmetric or asymmetric bulges between the DAs. Each synthetic RNA oligonucleotide can be designed to include 16-120 nucleotides. Each synthetic RNA oligonucleotide can be designed to include 1 or more modified nucleotides.

The rational design of the synthetic RNA oligonucleotides and/or RNA nanostructures described herein can be carried out in a computing environment. The computing environment can include one or more computing devices that can include at least one processor circuit, for example, that can have a processor and a memory. Various applications and/or other functionality may be executed in the computing environment according to various aspects of the disclosure. Also, various data can be stored in one or more data stores that can be accessible to the computing environment.

The components executed on the computing environment, for example, can include a rational RNA design system and other applications, services, processes, systems, engines, or functionalities not discussed in detail herein. The rational RNA design system can be executed to facilitate the design of synthetic RNA oligonucleotides and/or RNA nanostructures as described herein. The rational RNA design system can also perform various back end functions that can be associated with the design of RNA oligonucleotides, such as those synthetic RNA oligonucleotides and/or RNA nanostructures described herein.

The synthetic RNA oligonucleotides can be synthesized using standard molecular biologic and biochemical techniques. In other words, the various nucleic acids that can form the RNA nanoparticles can be de novo synthesized as desired. Such synthesis techniques will be known to the skilled artisan.

### Pharmaceutical Formulations

Also provided herein are pharmaceutical formulations that can include an amount of an RNA complex or nanostructure described herein and a pharmaceutical carrier appropriate for administration to an individual in need thereof. The individual in need thereof can have or can be suspected of a cancer or other disease or disorder in need of treatment or prevention. In some embodiments, the subject in need thereof is in need of a diagnostic procedure, such as an imaging procedure. The pharmaceutical formulations can include an amount of an RNA complex or nanostructure described herein that can be effective to treat or prevent a cancer.

Formulations can be administered via any suitable administration route. For example, the formulations (and/or compositions) can be administered to the subject in need thereof orally, intravenously, ocularly, intraocularly, intramuscularly, intravaginally, intraperitoneally, rectally, parenterally, topically, intranasally, or subcutaneously. Other suitable routes are described herein. In some embodiments, the RNA complex or nanostructure contains an effective amount of a cargo molecule.

The RNA complex or nanostructure can be formulated for parenteral delivery, such as injection or infusion, in the form of a solution or suspension. The formulation can be administered via any route, such as, the blood stream or directly to the organ or tissue to be treated.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the RNA nanostructures as described herein can be prepared in water or another solvent or dispersing medium suitably mixed with one or more pharmaceutically acceptable excipients including, but not limited to, surfactants, dispersants, emulsifiers, pH modifying agents, and combinations thereof.

Suitable surfactants can be anionic, cationic, amphoteric or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the RNA complex or nanostructure.

The formulation can be buffered to a pH of 3-8 for parenteral administration upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the RNA complexes or nanostructures in the required amount in the appropriate solvent or dispersion medium with one or more of the excipients listed above, as required, followed by filtered sterilization. Dispersions can be prepared by incorporating the various sterilized RNA complexes or nanostructures into a sterile vehicle which contains the basic dispersion medium and the required other ingredients from those listed above. Sterile powders for the preparation of sterile injectable solutions can be prepared by vacuum-drying and freeze-drying techniques, which yields a powder of the RNA complexes or nanostructures plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of particles formed from one or more RNA complexes or nanostructures. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other aspects, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for use of nanotechnology including nanoformulations for parenteral administration can also contain one or more excipients, such as dispersing agents, wetting agents, and suspending agents.

The RNA complexes or nanostructures as described herein can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some aspects, the RNA complexes or nanostructures can be administered as a liquid formulation, such as a solution or suspension, a semi-solid formulation, such as a lotion or ointment, or a solid formulation. In some aspects, the RNA nanostructures can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some aspects, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some aspects, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some aspects, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some aspects, the surfactant can be a non-ionic surfactant. In other aspects, the emulsifying agent is an emulsifying wax. In further aspects, the liquid non-volatile non-aqueous material is a glycol. In some aspects, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing RNA nanostructures as described herein are also provided. In some aspects, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing an RNA complexes or nanostructures as described herein are also provided. The cream can contain emulsifying agents and/or other stabilizing agents. In some aspects, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some aspects of a cream formulation, the water-base percentage can be about 60% to about 75% and the oil-base can be about 20% to about 30% of the total, with the other percentages being the emulsifier agent, preservatives and additives for a total of 100%.

Ointments containing an RNA complexes or nanostructures as described herein and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing an RNA complexes or nanostructures as described herein, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alkylene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited to, isopropyl myristate, ethyl acetate, C₁₂-C₁₅ alkyl benzoates, mineral oil, squalane, cyclomethicone, capric/caprylic triglycerides, and combinations thereof.

Also described herein are foams that can include an RNA complexes or nanostructures as described herein. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of a composition. The buffers can buffer the composition from a pH of about 4 to a pH of about 7.5, from a pH of about 4 to a pH of about 7, or from a pH of about 5 to a pH of about 7. In some aspects, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain aspects, the formulations can be provided via continuous delivery of one or more formulations to a patient in need thereof. For topical applications, repeated application can be done or a patch can be used to provide continuous administration of the noscapine analogs over an extended period of time.

The RNA complexes or nanostructures as described herein can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art.

Formulations containing RNA complexes or nanostructures as described herein can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing an RNA complexes or nanostructures as described herein can be prepared using one or more pharmaceutically acceptable excipients, including diluents, preservatives, binders, lubricants, disintegrators, swelling agents, fillers, stabilizers, and combinations thereof.

Delayed release dosage formulations containing an RNA complexes or nanostructures as described herein can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing an RNA complexes or nanostructures as described herein can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

In some aspects, an amount of one or more additional active agents are included in the pharmaceutical formulation containing an RNA complex or nanostructure. Suitable additional active agents include, but are not limited to, DNA, RNA, modified ribonucleotides, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics (anti-cancer drugs). Other suitable additional active agents include, sensitizers (such as radiosensitizers). The RNA nanostructure can be used as a monotherapy or in combination with other active agents for treatment or prevention of a disease or disorder.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylae, and sodium salicaylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, aspargainase erwinia chyrsanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, all-trans retinoic acid, and other anti-cancer agents listed elsewhere herein.

### Methods of Using the RNA Complexes and Nanostructures and Formulations Thereof

The RNA complex or nanostructure as provided herein can be administered to a subject in need thereof, cell, or population thereof. The subject in need thereof can have a cancer or any other disease or disorder that would benefit from a nucleoside analogue. The amount delivered can be an effective amount of an RNA complex or nanostructure provided herein. The subject in need thereof can be symptomatic or asymptomatic. In some aspects, the RNA complexes or nanostructures provided herein can be co-administered with another active agent. It will be appreciated that co-administered can refer to an additional compound that is included in the formulation or provided in a dosage form separate from the RNA complex or nanostructure or formulation thereof. The effective amount of the RNA complex or nanostructure or formulation thereof, such as those described herein, can range from about 0.1 mg/kg to about 500 mg/kg. In some aspects, the effective amount ranges from about 0.1 mg/kg to 10 mg/kg. In additional aspects, the effective amount ranges from about 0.1 mg/kg to 100 mg/kg. If further aspects, the effective amount ranges from about 0.1 mg to about 1000 mg. In some aspects, the effective amount can be about 500 mg to about 1000 mg.

Administration of the RNA complex or nanostructure and formulations thereof can be systemic or localized. The compounds and formulations described herein can be administered to the subject in need thereof one or more times per day. In an aspect, the compound(s) and/or formulation(s) thereof can be administered once daily. In some aspects, the compound(s) and/or formulation(s) thereof can be administered given once daily. In another aspect, the compound(s) and/or formulation(s) thereof can be administered twice daily. In some aspects, when administered, an effective amount of the compounds and/or formulations are administered to the subject in need thereof. The compound(s) and/or formulation(s) thereof can be administered one or more times per week. In some aspects the compound(s) and/or formulation(s) thereof can be administered 1 day per week. In other aspects, the compound(s) and/or formulation(s) thereof can be administered 2 to 7 days per week.

In some aspects, the RNA complex or nanostructure and/or formulation(s) thereof, can be administered in a dosage form. The amount or effective amount of the compound(s) and/or formulation(s) thereof can be divided into multiple dosage forms. For example, the effective amount can be split into two dosage forms and the first dosage form can be administered, for example, in the morning, and the second dosage form can be administered in the evening. Although the effective amount is given over two doses, in one day, the subject receives the effective amount. In some aspects the effective amount is about 0.1 to about 1000 mg per day. The effective amount in a dosage form can range from about 0.1 mg/kg to about 1000 mg/kg. The dosage form can be formulated for oral, vaginal, intravenous, transdermal, subcutaneous, intraperitoneal, or intramuscular administration. Preparation of dosage forms for various administration routes are described elsewhere herein.

The modular RNA motifs described herein and the RNA complexes or nanostructures described herein can be used in the preparation of a medicament for treatment of a disease or a cancer.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

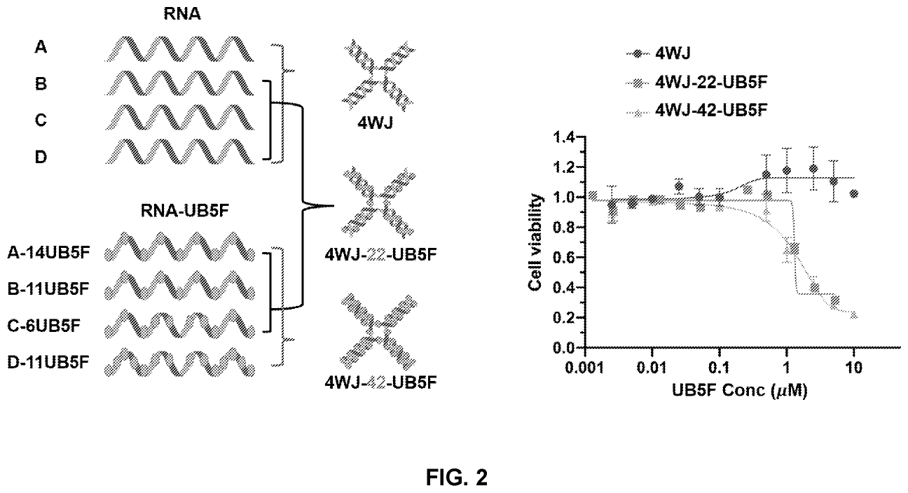

FIG. 2 shows results of a cytotoxicity study of RNA-UB5F nanoparticles in breast cancer cell line.

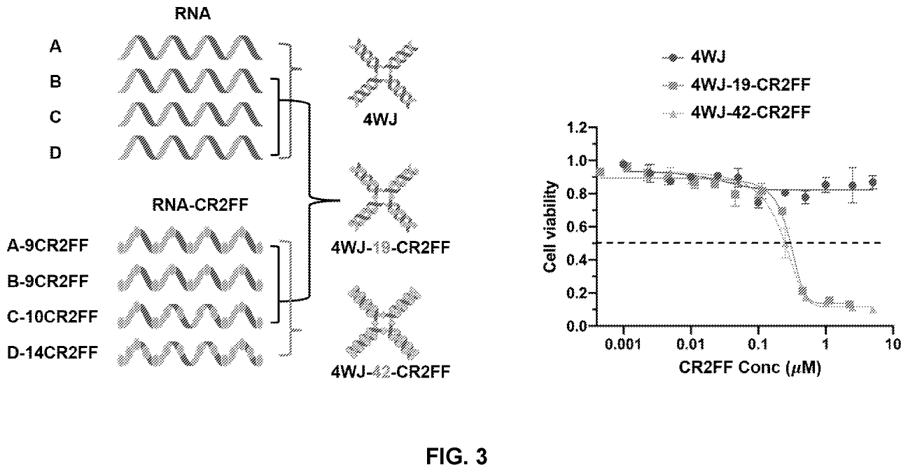

FIG. 3 shows results of a cytotoxicity study of RNA-CR2FF nanoparticles in breast cancer cell line.

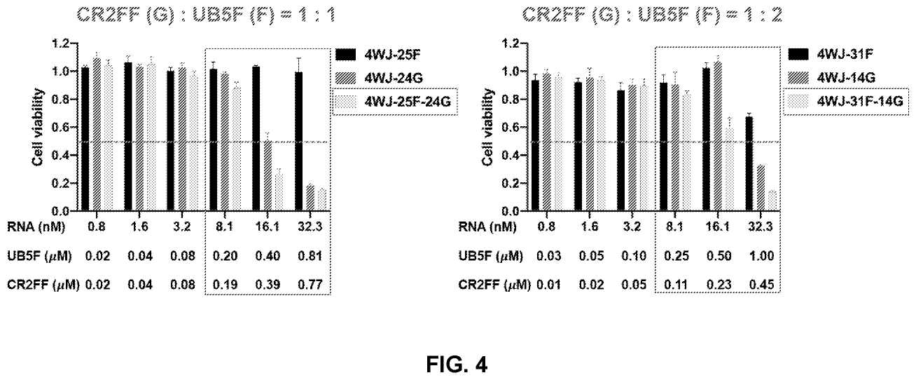

FIG. 4 shows results of a cytotoxicity study of RNA-CR2FF-UB5F nanoparticles in breast cancer cell line

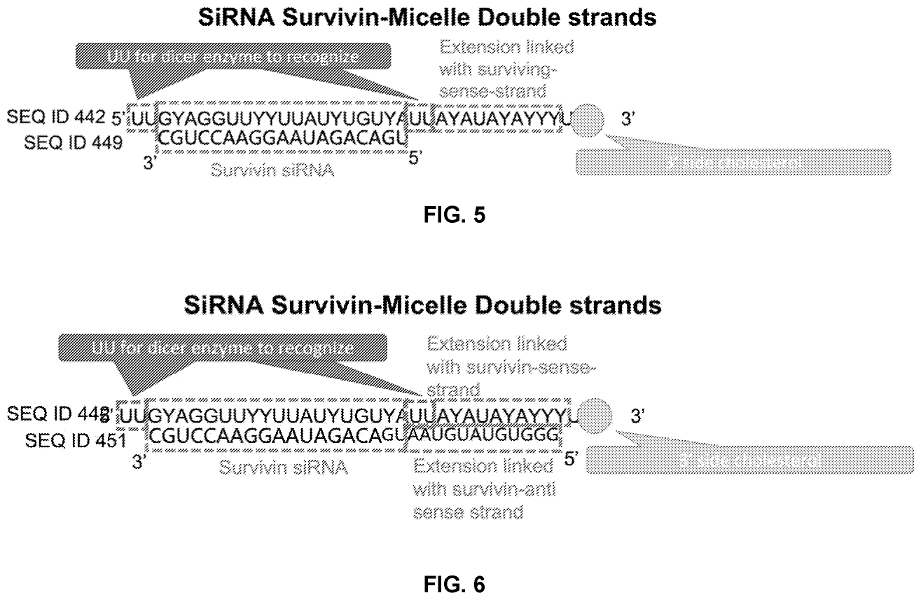

FIG. 5 shows Survivin-Double strands structure: Sense-Strand with 3′ cholesterol and gemcitabine (as Y)

FIG. 6 shows Survivin-Double strands structure: Sense-Strand with 3′ cholesterol and gemcitabine (as Y) and Anti sense-Strand with extension linked to it

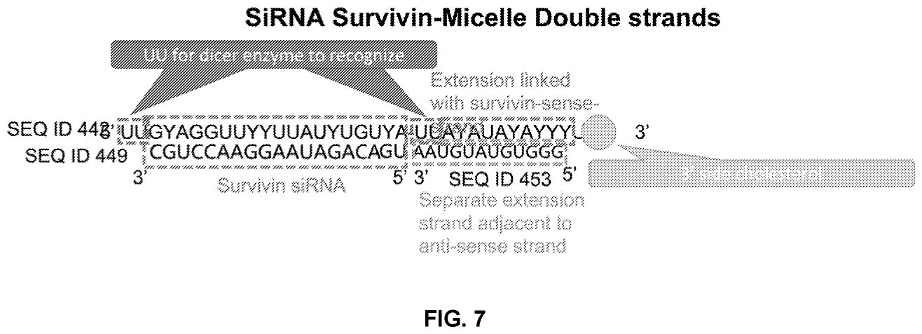

FIG. 7 shows Survivin-Double strands structure: Sense-Strand with 3′ cholesterol and gemcitabine (as Y) and Anti sense-Strand has an adjacent extension RNA structure

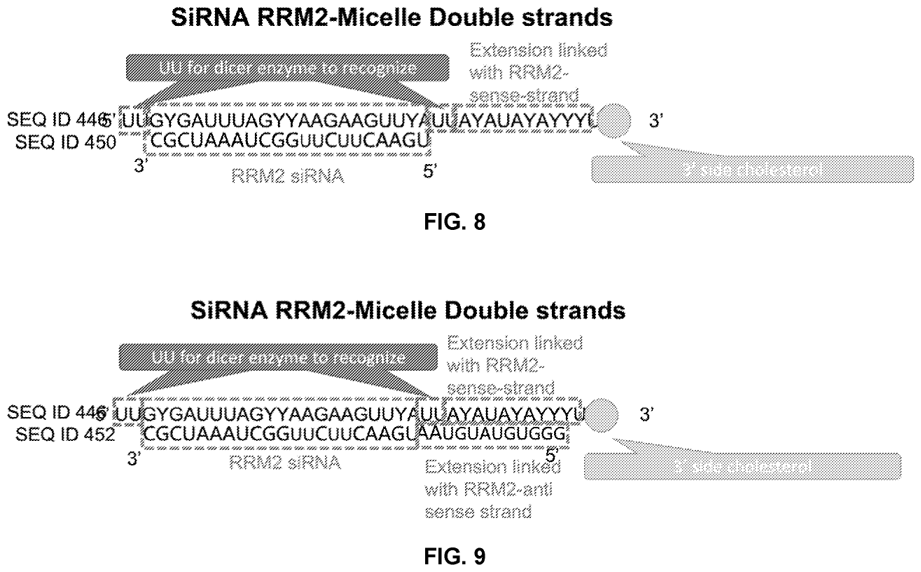

FIG. 8 shows RRM2-Double strands structure: Sense-Strand with 3′ cholesterol and gemcitabine (as Y)

FIG. 9 shows RRM2-Double strands structure: Sense-Strand with 3′ cholesterol and gemcitabine (as Y) and Anti sense-Strand with extension linked to it

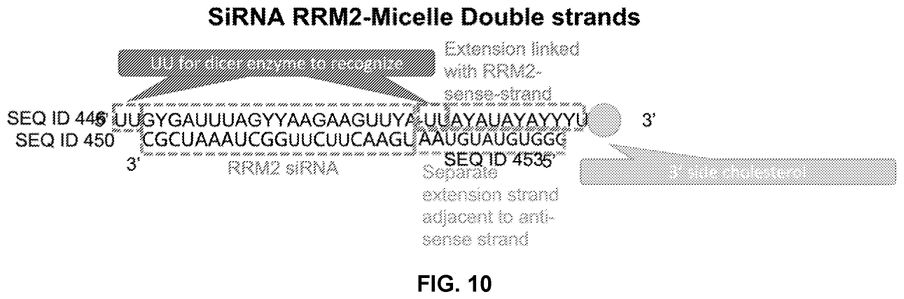

FIG. 10 shows RRM2-Double strands structure: Sense-Strand with 3′ cholesterol and gemcitabine (as Y) and Anti sense-Strand has an adjacent extension RNA structure

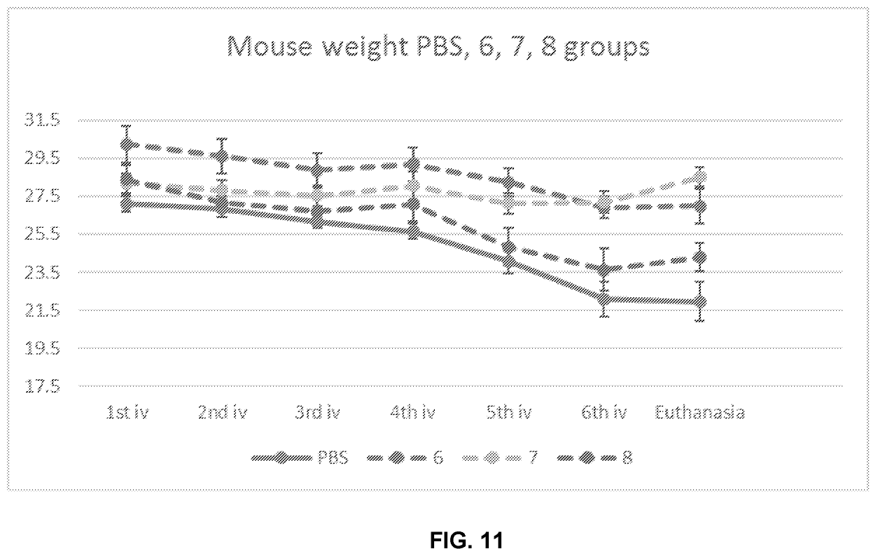

FIG. 11 Curves showing results of a weight change study of RNA-CR2FF-Cholesterol nanoparticles in human colorectal adenocarcinoma cell lung metastasis animal model.

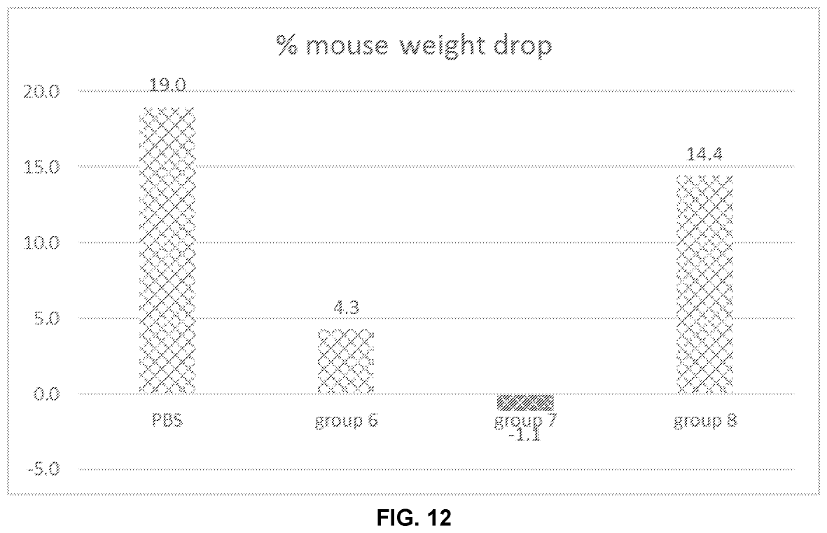

FIG. 12 Histogram showing results of a weight change study of RNA-CR2FF-Cholesterol nanoparticles in human colorectal adenocarcinoma cell lung metastasis animal model.

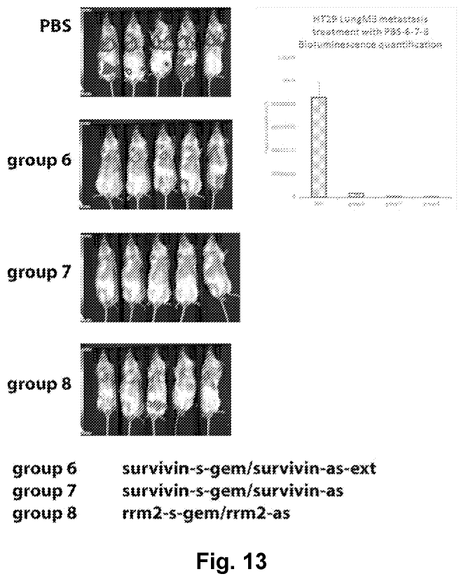

FIG. 13 shows results of in vivo metastasis inhibition study of RNA-CR2FF-Cholesterol nanoparticles in human colorectal adenocarcinoma cell lung metastasis animal model.

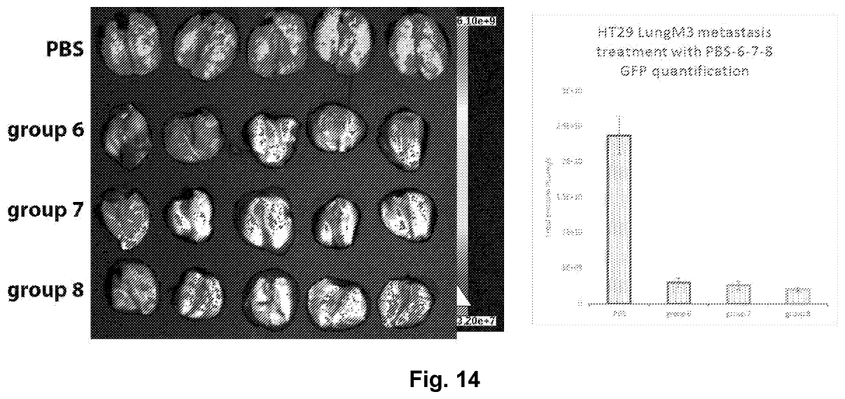

FIG. 14 shows results of ex vivo metastasis inhibition study of RNA-CR2FF-Cholesterol nanoparticles in human colorectal adenocarcinoma cell lung metastasis animal model.

### Example 2: Efficient Inhibition of Lung CRC Metastasis by Nontoxic RNA Micelles Carrying Both siRNA and Gemcitabine

### Materials and Methods

### Synthesis of RNA Micelles

RNA-micelles harboring gemcitabine and either survivin or RRM2 siRNA were constructed using a one-pot single-step assembly. Each micelle consisted of two component strands: 1) cholesterol-gemcitabine-sense siRNA and 2) anti-sense siRNA. RNA strands were synthesized using solid-phase synthesis and with commercially available phosphoramidite monomers of 2′-TBDMS Adenosine (n-bz) CED, 2′-TBDMS Guanosine (n-ibu) CED, N4-Benzoyl-2′-deoxy-5′-O-DMT-2′,2′-difluorocytidine 3′-CE phosphoramidite (Gemcitabine amidite), and 2′-Fluoro Uridine CED, followed by deprotection according to manufacturer (Azco Biotech) provided protocol. Cholesterol was attached to the 3′-end of sense siRNA strand by using 3′-Cholesteryl-TEG CPG support (Glen Research Corp.), following manufacturer instructions. Alexa-fluor 647 label was included into micelles on the cholesterol-gemcitabine-sense siRNA strand. Conjugation reactions were carried out by mixing a 1:10 molar ratio of primary amine labeled RNA:NHS ester-fluorophore in 0.1 M sodium bicarbonate buffer, pH=8.5. The conjugation reactions were incubated at room temperature for 16 h while protected from light, as previously described (Ghimire, C., et al. ACS Nano 2020, 14:13180-13191). Synthesized RNA is purified from terminated strands using an ion-pair reverse phase column which typically yields complete labeling with cholesterol during solid phase synthesis.

Sequences:
Cholesterol-gemcitabine-survivin sense
(SEQ ID NO: 274)
5′-XXGYAGGXXYYXXAXYXGXYAXXAYAXAYAYYYX-3′ - Cholesterol,
where Y is gemcitabine and X is 2′-F Uridine (floxuridine).
Survivin anti-sense
(SEQ ID NO: 1)
5′-UGACAGAUAAGGAACCUGC-3′
Survivin anti-sense-extension
(SEQ ID NO: 275)
5′- GGGXGXAXGXAAUGACAGAUAAGGAACCUGC-3′
wherein X is 2′-F Uridine (floxuridine).
Cholesterol-gemcitabine-RRM2 sense
(SEQ ID NO: 276)
5′- XXGCGAXXXAGCCAAGAAGXXCAXXACAXACACCCX-3′,
where Y is gemcitabine and X is 2′-F Uridine (floxuridine).
RRM2 anti-sense
(SEQ ID NO: 2)
5′- UGAACUUCUUGGCUAAAUCGC-3′
Cholesterol-gemcitabine-survivin sense-Alexa647
(SEQ ID NO: 277)
5′ Alexa647-XXGYAGGXXYYXXAXYXGXYAXXAYAXAYAYYYX-3′ Cholesterol,
where Y is gemcitabine and X is 2′-F Uridine (floxuridine).
Gemcitabine-survivin sense
(SEQ ID NO: 278)
5′- XXGYAGGXXYYXXAXYXGXYAXXACAXAYAYYYX-3′,
where Y is gemcitabine and X is 2′-F Uridine (floxuridine).
Survivin sense
(SEQ ID NO: 279)
5′- GCAGGXXCCXXAXCXGXCAXX-3′,
where Y is gemcitabine and X is 2′-F Uridine (floxuridine).

The two component micelle strands were assembled into micelles through mixing at equal molar concentrations in TMS buffer (50 mM Tris pH=8.0, 100 mM NaCl, 10 mM MgCl₂) or PBS buffer (137 mM NaCl, 2.7 mM KCl, 10 mM Na₂HPO₄ and 2 mM KH₂PO₄, pH 7.4) followed by heating to 95° C. for 5 min and slowly cooling to 4° C. over the course of 45 min, as previously described (Shu, Y., et al. J. Control. Release 2018, 276:17-29; Yin, H., et al. ACS Nano 2019, 13:706-717; Guo, S., et al. Nat. Commun. 2020, 11:972-982; Li, H., et al. Adv. Mater. 2016, 28:7501-7507; Khisamutdinov, E. F., et al. Nucleic Acids Res. 2014, 42:9996-10004).

### Characterization of RNA Micelles

RNA-micelles were assayed for formation via agarose gel electrophoresis using 1% agarose gel ran at 4° C., 100 V for 30 mins on TAE running buffer.

Apparent hydrodynamic sizes and zeta potential of pre-assembled RNA-micelles was measured. All RNA samples were measured in Diethylpyrocarbonate H₂O and PBS buffer (137 mmol/I NaCl, 2.7 mmol/I KCl, 100 mmol/1 Na₂HPO₄, 2 mmol/1 KH₂PO₄, pH 7.4) at 25° C.

### Cell Culture

Human CRC cell lines HT29, HT29 G-L LungM3 were grown and cultured in medium containing both 10% fetal bovine serum and penicillin/streptomycin in a 37° C. incubator with a 5% CO₂ and a humidified atmosphere.

### In Vitro Proliferation Assay of NRA Micelles

Cell cytotoxicity was studied using CellTiter 96 Non-Radioactive Cell Proliferation Assay (Promega) following manufacturer instructions. Briefly, 5×10³ HT29 cells were seeded on a 96-well plate overnight. RNA micelles, free gemcitabine, and siRNA were added into each well in triplicates at 5, 25, 100, and 250 nM of gemcitabine concentrations. After incubation at 37° C. for 48 h in a humidified 5% CO₂ environment, 15 μL of MTT Dye Solution was added to each well and the plate was incubated at 37° C. in the dark for 4 h. Next, 50 μL Solubilization Solution/Stop Mix was added to each well for dissolving the crystal in the dark. The plate was incubated for 2 h at room temperature. Finally, the crystal in each well were fully dissolved to a uniformly colored solution and their absorbance at 570 nm was measured by Synergy 4 microplate reader (Bio-Tek). Data were collected by Gen5, and normalized data was plotted using Origin.

### In Vivo Tumor Development

Female and male NOD.Cg-Prkdc scid Il2rgtm1Wjl/SzJNCr nude (8 wks old) mice were obtained from Jackson Labs and housed in clean, pathogen-free rooms in an environment with controlled temperature (27° C.), humidity, and a 12 h light/dark cycle. The mice were fed standard chow and tap water ad libitum and allowed to acclimate for 1 week. All animal experiments were approved by the Institutional Animal Care and Use Committee at the University of Kentucky and were conducted in accordance with guidelines issued by the National Institutes of Health for the care of laboratory animals. HT29 cells were trained to be lung tropic through initial IV injection. Lung metastases were harvested and re-injected into mice; the in vivo selection process which is repeated three times to develop the HT29 G-L LungM3 cell line. HT29 G-L LungM3 tumor cells were injected iv into mice for lung metastasis development. Mice were anesthetized using isoflurane (2% in oxygen at 0.6 L/min flow rate) and injected with 1×10⁶ cells in 100 μL of PBS.

### In Vivo Tumor Regression of CRC Metastasis Tumors

NOD.Cg-Prkdc scid Il2rgtm1Wjl/SzJNCr nude mice with confirmed HT29 G-L LungM3 tumors were treated with samples (including PBS, micelle-extension/siSurv, micelle/siSurv, micelle/siRRM2 twice a week at 1 mg/kg dose in 300 μl by tail-vein injection. Mice were treated a total of 6 time. On day 26, were euthanized and imaged for bioluminescent and GFP signal with Lago SII (Spectral Instruments Imaging, Tucson, AZ). Tumors of the mice were collected and fixed in 10% formaldehyde and embedded in paraffin blocks. Tumor sections were stained for Ki-67 (Cell Signaling #9027) or IHC cleaved Caspase 3 (Cell signaling #9579) primary antibody followed by fluorochrome-conjugated secondary antibodies. Tumor sections were then microscopically imaged.

### Chemokine Induction of RNA Micelles by ELISA

RAW 264.7 cells were plated into 24-well plates with the density of 2.5×10⁵ cells per well and cultured overnight. Then, RNA micelles were diluted in Opti-MEM medium (Life Technologies Corporation, Carlsbad, CA, USA) and added to the cells. The cells were continually cultured for 8 h at 37° C. in humidified air containing 5% CO₂, and the cell culture supernatant were collected and stored at −80° C. until use. The concentration of TNF-α and IL-6 in the supernatant were determined by enzyme-linked immunosorbent assay (ELISA) using Mouse ELISA MAX™ Deluxe sets (BioLegend, Inc., San Diego, CA), following protocols provided by the manufacturer.

### Results

RNA-Micelles Harbor Numerous Copies of Chemical Drug and siRNA by Forming into Homogenous Micellular Structures.

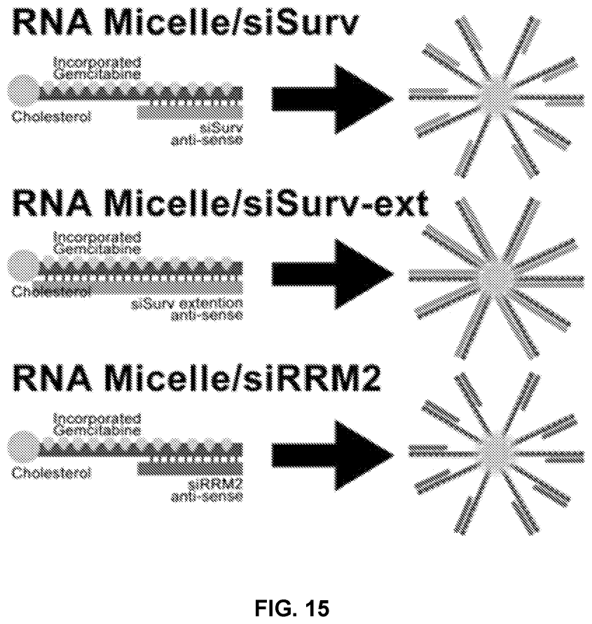

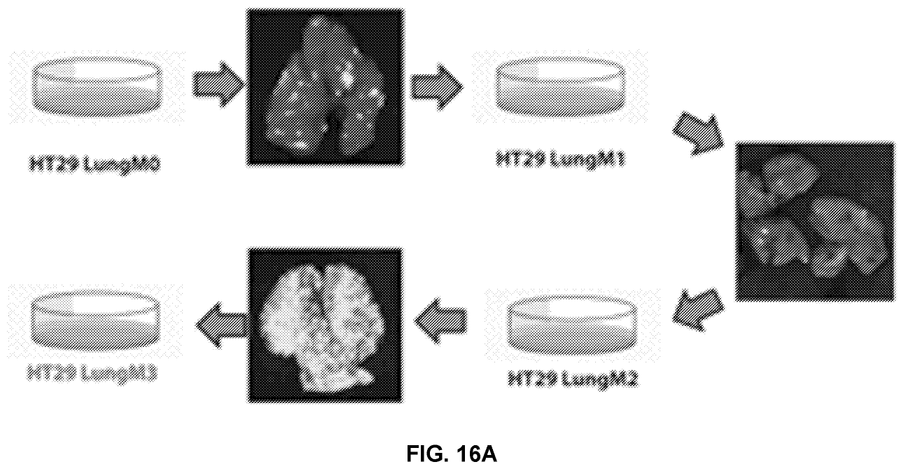

RNA-micelles were previously developed using the three-way junction from the pRNA (pRNA-3WJ) of the Phi29 DNA packaging motor by including a cholesterol tag on the 3′ end of one of the component strands (Shu, Y., et al. J. Control. Release 2018, 276:17-29; Yin, H., et al. ACS Nano 2019, 13:706-717). These micelles were conjugated with paclitaxel or anti-miRNA sequences for delivery to in vivo tumor models in mice. Here, RNA-micelles were redesigned into a more simplistic structure that self-assembled from cholesterol siRNA chimeras as seen in FIG. 15. Furthermore, gemcitabine was included into the micelle design through nucleotide replacement for cytosine ribonucleotides. Each RNA chimera was designed to include twelve copies of gemcitabine on the sense strand of survivin and RRM2 siRNA. Thus, during gemcitabine release, the anti-sense siRNA is also released to RISC without Dicer processing. RNA-micelle components were synthesized via solid-phase synthesis using phosphoramidite technology; cholesterol via 3′-Cholesteryl-TEG CPG and gemcitabine via Gemcitabine amidite were placed on the sense strand of the siRNA. Upon purification of the two RNA strands, they were folded through an annealing step in which the siRNA would fold, and the cholesterol tags would self-assemble into a hydrophobic core forming micelles.

RNA-micelles were then characterized for formation and physical characteristics. First micelle formation was confirmed through 1% Agarose gel compared to single stranded components. From the assembly gel, cholesterol-sense strands self-assembled into a micellular structure as indicated by the relatively slow migration rate, this micellular structure is maintained as the anti-sense siRNA strand is annealed with the sense strand-micelle. As shown in the agarose gel, the RNA-micelles form into a single band showing formation of a homogenous product. Additionally, dynamic light scattering (DLS) was completed on the resulting While these RNA-micelles are larger than most RNA nanoparticles created (Ghimire, C., et al. ACS Nano 2020, 14:13180-13191; Guo, S., et al. Nat. Commun. 2020, 11:972-982; Li, H., et al. Adv. Mater. 2016, 28:7501-7507; Khisamutdinov, E. F., et al. Nucleic Acids Res. 2014, 42:9996-10004), they still fall within the nanometer range and retain their inherent properties of RNA nanoparticles.

RNA-Micelles are Processed by DNase I/for the Specific Release of Gemcitabine which in Turns Releases siRNA Anti-Sense Strands.

Gemcitabine has previously been incorporated into nucleic acid sequences for improved therapeutic delivery to tumor cells (Zhu, L., et al. J Am Chem Soc 2022, 144:1493-1497; Ma, Y., et al. Chem Commun (Camb) 2019, 55:6603-6606; Pan, G., et al. ACS Appl Mater Interfaces 2019, 11:41082-41090). These nucleic acid structures, including drugtamer and nanogels, has demonstrated the ability to cleave gemcitabine through incubation with DNases. Specifically, DNase II was able to release gemcitabine, and contains high concentrations in the lysosome (Zhu, L., et al. J Am Chem Soc 2022, 144:1493-1497). To demonstrate stability and controlled release of gemcitabine and survivin or RRM2 siRNA from the RNA-micelles, the nanoparticles were incubated with fetal bovine serum (FBS) containing an array of nucleases or DNase II. After time-course incubation, RNA micelle structures were assayed by native PAGE. Interestingly, RNA-micelles demonstrated stability against FBS degradation, while showing degradation and thus release of gemcitabine and the anti-sense siRNA strand. As shown in the gel the RNA-micelles were degraded by the DNase II, thus resulting in short RNA fragments at the bottom of the gel and a single band at ˜20 nt indicating the anti-sense survivin and RRM2 strands were released, while remaining stable. The results indicate that the RNA-micelles are able to remain stable without premature release of either gemcitabine or siRNA while circulating through the body but will be processed within the cell for control release of the therapeutics intracellularly.

RNA-Micelles Spontaneously Target and Internalize into CRC Cells.

While gemcitabine efficiently penetrates across cell membranes using nucleoside transporters, tumors cells quickly develop resistance by down regulating such nucleoside transporters such as SLC29A1 SLC28A1, and SLC28A3. Cells lacking nucleoside transporters do not allow for gemcitabine influx and are not susceptible to gemcitabine's cytotoxic nature. Additionally, nanoparticles rely on endocytosis as an internalization mechanism that often results in endosome trapping thus limiting therapeutic efficacy. To test the internalization of the RNA-micelles for intracellular delivery of gemcitabine and siRNA, Alexa-fluor 647 fluorescent tags were included in RNA-micelle designs. RNA-micelle/Alexa647 were then incubated with HT29 and HCT116 CRC cell lines and imaged by live cell confocal microscopy. Over time course imaging, confocal microscopy demonstrated an increase in fluorescent signaling within the CRC cells indicating the RNA-micelles were able to bind and internalize into cells. As the RNA-micelles do not contain CRC targeting ligands, this binding is spontaneous and does not rely on receptor mediated endocytosis. We hypothesize that the large cholesterol content of the RNA-micelles allows for interactions with cholesterol rich lipid rafts on the cell membrane, thus leading to raft-dependent endocytosis (EI-Sayed, A., et al. Mol Ther 2013, 21:1118-1130). This endocytosis pathway is cholesterol-dependent and can or cannot involve caveolae (Lajoie, P., et al. Int Rev Cell Mol Biol 2010, 282:135-163; Lajoie, P., et al. J Cell Mol Med 2007, 11:644-653). To test if the RNA-micelles are endocytosed, confocal microscopy studies were repeated while staining cells (Zheng, Z., et al. J. Control. Release 2019, 311-312:43-49). Results demonstrated. While endosome trapping can inhibit drug delivery DNase II is found in high levels in the lysosome, thus processing the release of gemcitabine and siRNA without Dicer processing allowing the therapeutics to remain efficacious (see below) (Zhu, L., et al. J Am Chem Soc 2022, 144:1493-1497).

### RNA-Micelles do not Result in Immune Responses.

Cancer therapeutics, including chemotherapies and nanoparticle platforms, have commonly shown not only toxicities through interactions with healthy tissues but been known to elicit immune responses through cytokine induction (Lee, C. S., et al. World J Gastroenterol 2014, 20:3751-3761; Driscoll, J., et al. Curr Protoc 2021, 1:e249; Hong, E., et al. Nano Lett. 2018, 18:4309-4321). RNA nanoparticles have previously been shown to control immune responses through nanoparticle size, shape, and sequence (Khisamutdinov, E. F., et al. Nucleic Acids Res. 2014, 42:9996-10004; Guo, S., et al. Mol. Ther. Nucleic Acids 2017, 9:399-408). Thus RNA nanoparticles can be designed to induce or avoid immune responses. Furthermore, the conjugation of paclitaxel to RNA nanoparticles resulted in a significant decrease in cytokine induction (Guo, S., et al. Nat. Commun. 2020, 11:972-982). RNA-micelles were incubated with RAW 264.7 macrophage cells and assayed for cytokine responses by Elisa. Results demonstrated that control RNA-micelle as well as RNA-micelles harboring gemcitabine and survivin or RRM2 siRNA did not result in significant increase of cytokine induction compared to cell only samples. These results demonstrate the RNA-micelles are safe against activating large immune responses and allows for in vivo compatibility.

RNA-Micelles Specifically Inhibit CRC Lung Metastases without Adverse Effects on the Body

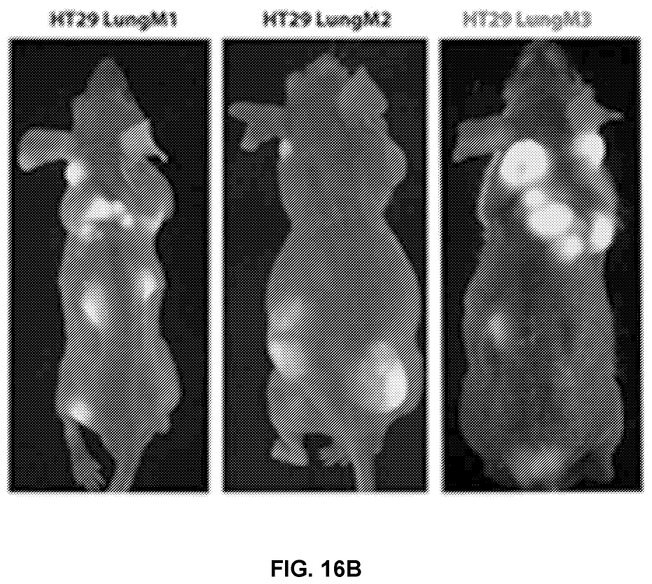

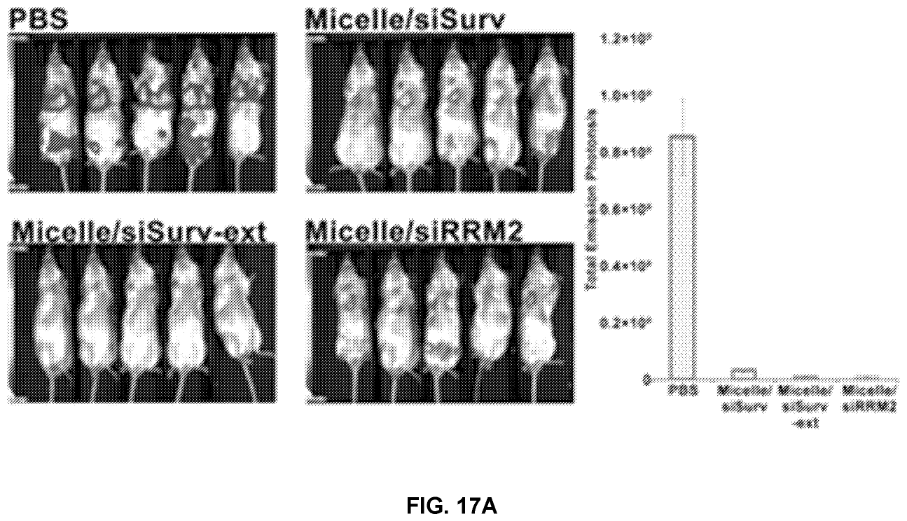

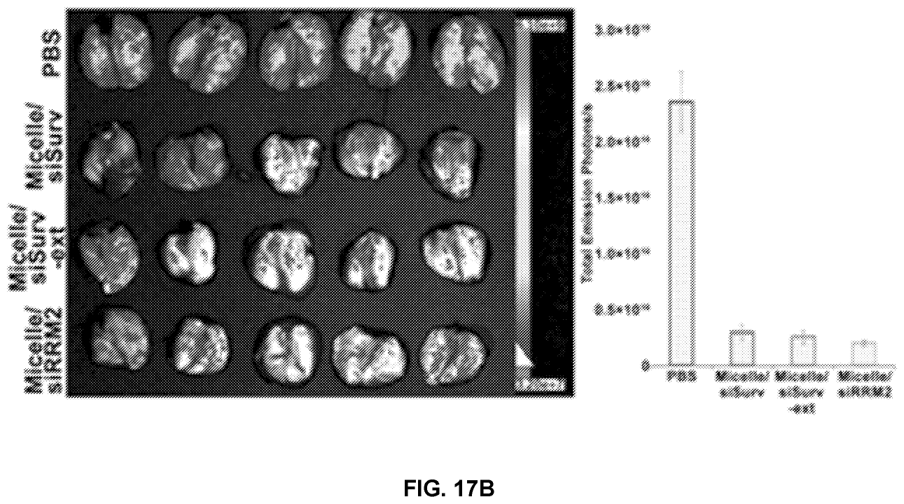

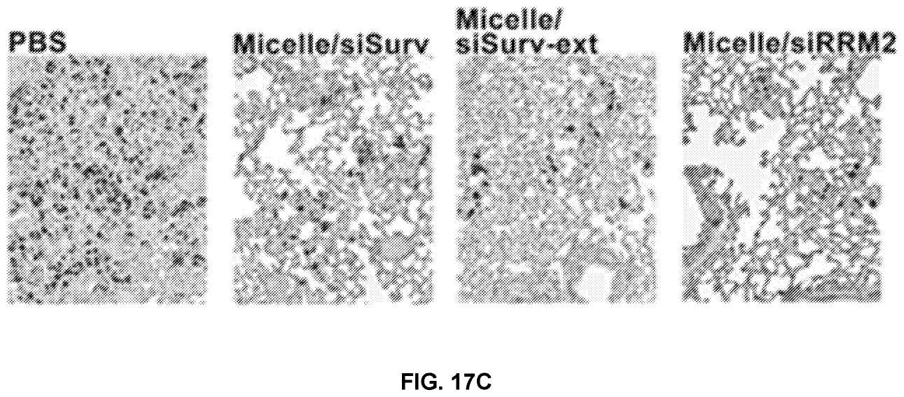

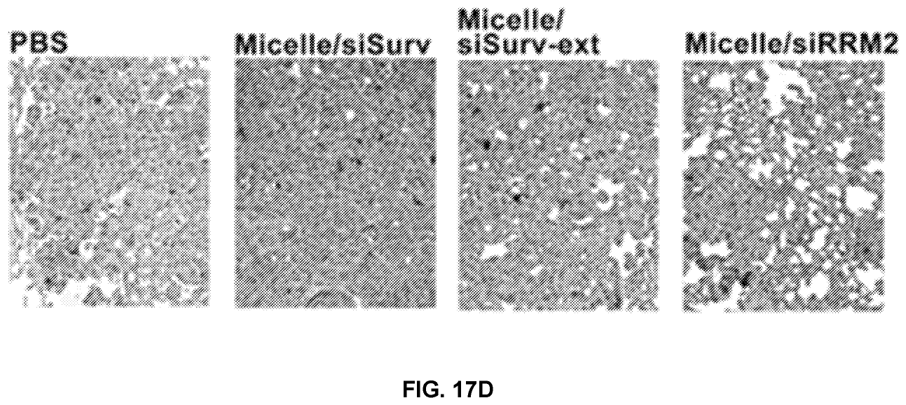

The RNA-micelles demonstrated effective CRC inhibition and the ability to deliver high levels of gemcitabine and either survivin or RRM2 siRNA. Developed RNA-micelles were tested for their ability to inhibit CRC lung metastasis growth in mouse models. HT29 G-L LungM3 cells, a derivative of HT29 CRC cells that have been “trained” to be lung tropic (Reichel, D., et al. Pharm Res 2017, 34:2385-2402; Rychahou, P., et al. J Control Release 2018, 275:85-91), were injected intravenously into nude mice to generate tumors on the lungs of the mice. The HT29 G-L LungM3 cells express GFP to allow for monitoring of tumor growth and regression (FIGS. 16A and 16B). Once tumors were developed RNA micelles were delivered over 6 treatments to mice on a twice a week schedule. At the conclusion of the nanoparticle regimen, mice were imaged by IVIS, and lungs were excised and imaged. Results demonstrate significant decrease in GFP signaling in both image sets demonstrating regression of lung tumors (FIGS. 17A and 17B. Interesting the RNA-micelles harboring gemcitabine and survivin siRNA particles resulted in almost an entire elimination of GFP signaling, hinting at complete treatment of the tumors. To further examine the treatment of the mice, lungs were cryo-sectioned and stained for Ki-67 and Caspase 3 signaling (FIGS. 17C and 17D). Ki-67 signaling of the treatment groups demonstrated some signaling throughout the lungs indicating the presence of micro-tumors still existing. Furthermore, lungs demonstrated low levels of caspase signaling indicating tumor death was not a result of caspase pathways. Overall, dosing of RNA-micelles can further be optimized, or tumor could be treated with a longer regimen to lead to complete tumor regression. Yet the presented data indicate the RNA-micelles are quite effective in treating CRC lung metastasis. During the in vivo experiments, the weight of the mice was monitored. Results indicate no significant weight loss was seen in the RNA-micelle samples carrying survivin or RRM2 siRNA compared to PBS groups indicating no obvious sign of toxicity to the mice. This data combined with the lack of cytokine induction indicate the RNA-micelles are generally safe and compatible for in vivo studies. However, further safety studies are required on the RNA-micelles to moves towards the clinic.

### Discussion

Nanoparticles often are complex structures that necessitate several synthesis or chemistry steps to form a completed particle with conjugated drugs. As a result, product yield decreases, cost of production and labor increases, and nanoparticle quality can suffer from lack of homogeneity. The developed RNA-micelles presented here overcome these issues using a single-step, one-pot production method that results in the production of homogenous and stable micelles. During the solid-phase synthesis of the component RNAs, gemcitabine was directly incorporated through the use of phosphoramidites that has a yield of 98.5% incorporation; thus preventing further chemical conjugation steps. Furthermore, RRM2 and survivin siRNA us simply incorporated through sequence extension from the RNA-micelle and simple hybridization with the anti-sense strand. As a result, the presented RNA micelles are very simplistic in design, can be synthesized with ease and self-assemble through nucleic acid folding and cholesterol interactions. The RNA-micelles proved to remain stable against nucleases and form into a homogeneous product with a small size distribution.

As previously shown, RNA is a known elastomer that is able to change size and shape under external forces (Bao, L., et al. Biophys. J. 2017, 112:1094-1104; Kriegel, F., et al. J. Struct. Biol. 2017, 197:26-36; Lipfert, J., et al. Proc. Natl. Acad. Sci. U.S.A. 2014, 111:15408-15413; Chou, F. C., et al. PLoS Comput. Biol. 2014, 10:e1003756). Furthermore, we have shown that our 10 nm RNA nanoparticles can be stretched under force to oblong structure and return to its original shape and size upon force relaxation (Ghimire, C., et al. ACS Nano 2020, 14:13180-13191). Combining these results with the fact that RNA is dynamic in nature, and continually restructuring (Binzel, D. W., et al. Chem Rev 2021, 121:7398-7467; Li, X., et al. Adv Drug Deliv Rev 2022, 186:114316; Yu, A. M., et al. Mol Cell 2021, 81:870-883 e810; Hurst, T., et al. RNA Biol 2021, 1-11; Jing, Z., et al. J. Chem. Theory Comput. 2019, 15:6422-6432; Larsen, K. P., et al. Cold Spring Harb Perspect Biol 2019, 11), RNA nanoparticles are deformative and dynamic allowing for spontaneous tumor accumulation due to penetrating through leaky tumor vasculature. Our results above demonstrate a high yield of spontaneous therapeutic delivery to lung metastatic tumors without the need of tumor targeting ligands. It is believed the RNA-micelles are able to deform size to penetrate leaky vasculature under the blood pressure and act like a ratchet once in the tumor microenvironment that is unable to return to the blood circulation. Thus, forming a very effective therapeutic delivery platform. Furthermore, motile and deformable RNA-micelles allow the non-tumor-accumulating RNA-micelles to rapidly clear through kidney glomerular filtration into the urine, resulting in low vital organ accumulation and fast body clearance. Our 10-nm RNA nanoparticle demonstrated it can pass the 5.5-nm cutoff-size kidney glomeruli for renal excretion within 30 min to 1 h after iv injection with no detectable toxicity (Ghimire, C., et al. ACS Nano 2020, 14:13180-13191; Guo, S., et al. Nat. Commun. 2020, 11:972-982). Thus generating a delivery platform with low toxicities as demonstrated by the lack of mouse body weight changes.

Once accumulated in the CRC lung metastasis through spontaneous targeting, the RNA-micelles have demonstrated the ability to internalize into CRC cells. This internalization allows for the delivery of gemcitabine and siRNAs with high levels of efficacy. While the exact method of RNA-micelle internalization is not understood, as the micelles do not harbor targeting ligands for receptor mediated endocytosis, it is hypothesized that the richness in cholesterol in the lipid rafts of the cells may result in interactions with the high levels of cholesterol in the core of the RNA-micelles (EI-Sayed, A., et al. Mol Ther 2013, 21:1118-1130; Cho, Y. Y., et al. Molecules 2020, 25; Tian, N., et al. J Biol Chem 2012, 287:44447-44463; Nabi, I. R., et al. J Cell Biol 2003, 161:673-677). Thus this interaction between micelle cholesterol and lipid raft cholesterol can allow for endocytosis of the RNA-micelles. Once the RNA-micelles or individual RNA nanoparticle of the micelle is internalized, the twelve copies of gemcitabine are released through automatic processing by DNase II (Zhu, L., et al. J Am Chem Soc 2022, 144:1493-1497; Ma, Y., et al. Chem Commun (Camb) 2019, 55:6603-6606). This careful design of RNA-micelles with gemcitabine incorporated as part of the RNA sequence then allow for the release of the anti-sense siRNA strand without Dicer processing. The gemcitabine is placed on the sense strand of the siRNA, and through its release degrades the sense strand, resulting in a release of the anti-sense siRNA to be incorporated into RISC. This simple design of the micelles results in efficient cellular internalization, drug release, and high efficacy.

Bringing together the data presented, RNA-micelles harboring multiple copies of gemcitabine and siRNA show a strong promise for the treatment of CRC metastasis with the ability to translate into other tumor and metastasis types. The RNA-micelles have demonstrated to be cause no obvious toxicities or immune responses while circulating throughout the body. This is most probably due to the rapid renal excretion of non-tumor accumulated RNA-micelles. Furthermore, the intracellular internalization of the RNA-micelles results in an automatic processing of siRNA and gemcitabine. Thus, the mechanism of action of gemcitabine does not rely on nucleoside transporters that can be used to develop chemoresistance as commonly seen when delivering gemcitabine. Additionally, the combination of survivin or RRM2 siRNA with gemcitabine delivery not only creates a potent combinatory therapy, but further diversifies the therapy against building up chemoresistance. RNA-micelles have demonstrated their distinct ability in treating CRC lung metastasis, a disease that is currently a challenge in the clinic. From here, future research is needed to continue to progress these RNA based nanoparticles into the clinic through in-depth preclinical studies.

### CONCLUSIONS

Since its conception, RNA nanotechnology has grown into a versatile field that continues to prove its ability in delivery of therapeutics including siRNA, miRNA, and small molecule chemical drugs. Here we developed novel RNA micelles through the inclusion of hydrophobic cholesterol on double stranded RNA sequences including multiple copies of gemcitabine and a copy of survivin or RRM2 siRNA. These novel RNA micelles allowed for automatic intracellular processing to release both gemcitabine and the anti-sense siRNA strand in a single step. As a result, the RNA micelles, upon spontaneous targeting to CRC lung metastases, were able to deliver high payloads of therapeutics resulting in almost a complete regression of tumor signals. This work proves a powerful therapeutic tool though a simple one-step synthesis that is easy to construct and use.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A therapeutic RNA complex comprising a plurality of synthetic double-stranded RNA oligonucleotides, each synthetic double-stranded RNA oligonucleotide comprising a sense strand and an antisense strand,
wherein the sense strand has a 5′ end and a 3′ end with more than 25 nucleotides and a cholesterol molecule conjugated to the 3′ end of the sense strand, and the 2′-positions of all the pyrimidines at the sense strand do not contain oxygen,
wherein the antisense strand has a 5′ end and a 3′ end with 18-25 normal nucleotides,
wherein the sense strand is more than 18 nucleotides and the 2′-position of all the pyrimidine in the sense strand contain two fluorine and do not contain oxygen,
wherein the antisense stand comprises an siRNA sequence with unmodified nucleotides,
wherein the sense strand is the same length or longer than the antisense strand,
wherein at least 95% of the nucleotides in the antisense strand are hybridized to complementary nucleotides of the sense strand, and
wherein the plurality of synthetic double-stranded RNA oligonucleotides are in an aggregate with the cholesterol molecules at the center.

2. The therapeutic RNA complex of claim 1, wherein the sense strand contains 2′-difluoro-deoxypyrimidine; floxuridine (5-fluorodeoxyuridine, UB5F) nucleotides, gemcitabine (2′, 2′-difluoro-2′-deoxycytidine, dFdC, CR2FF) nucleotides, or a combination thereof.

3. The therapeutic RNA complex of claim 1, further comprising GalNAc (N-acetylgalactosamine), UAMC-1110 (SP-13786; (S)—N-(2-(2-Cyano-4,4-difluoropyrrolidin-1-yl)-2-oxoethyl)quinoline-4-carboxamide), FA (Folate), or DCL (N—[N—[(S)-1,3-dicarboxypropyl]carbamoyl]-(S)-lysine) on the 5′ end of the sense strand.

4. The therapeutic RNA complex of claim 1, wherein the antisense strand is an anticancer siRNA affecting survival or apoptosis.

5. The therapeutic RNA complex of claim 4, wherein the antisense strand is a survivin siRNA or RRM2 siRNA.

6. The therapeutic RNA complex of claim 5, wherein the survivin siRNA comprises the nucleic acid sequence SEQ ID NO:1.

7. The therapeutic RNA complex of claim 6, wherein the RRM2 siRNA comprises the nucleic acid sequence SEQ ID NO:2.

8. A synthetic double-stranded RNA oligonucleotide comprising a sense strand having a 5′ end and a 3′ end, an antisense siRNA strand having a 5′ end and a 3′ end, wherein the sense strand comprises 3, 4, 5, 6, 7, 8, 9, 10, 11, 12, 13, 14, 15, 16, 17, 18, 19, 20, or more floxuridine (5-fluorodeoxyuridine, UBSF) nucleotides, gemcitabine (2′, 2′-difluoro-2′-deoxycytidine, dFdC, CR2FF) nucleotides, or a combination thereof.

9. An RNA nanoparticle comprising at least three synthetic RNA nucleotides coupled to each other, wherein the at least three synthetic RNA oligonucleotides form a centrd no:al core domain and at least three double-stranded arms arranged around the core domain and extending away from the central core domain, and wherein at least one of the synthetic RNA oligonucleotides comprise a nucleic acid sequence selected from the group consisting of SEQ ID NOs:1-273, or a variant thereof having at least 90% sequence identity to a nucleic acid sequence selected from the group consisting of SEQ ID NOs:1-273.

10. The RNA nanoparticle of claim 9, wherein at least one of the synthetic RNA oligonucleotides comprise a nucleic acid sequence selected from the group consisting of SEQ ID NOS:167-221, or a variant thereof having at least 90% sequence identity to a nucleic acid sequence selected from the group consisting of SEQ ID NOs: 167-221.

11. The RNA nanoparticle of claim 9, comprising a first synthetic RNA oligonucleotide having the nucleic acid sequence SEQ ID NOs:6-9, 61, 280-283, 112-116, or 167-171, a second synthetic RNA oligonucleotide having the nucleic acid sequence SEQ ID NOs:10-13, 62-66, 117-121, or 172-176, and a third synthetic RNA oligonucleotide having the nucleic acid sequence SEQ ID NOs:14-18, 67-71, 122-126, or 177-181.

12. The RNA nanoparticle of claim 9, comprising a first synthetic RNA oligonucleotide having the nucleic acid sequence SEQ ID NOs:21-25, 41-45, 72-76, 92-96, 127-131, 147-151, 182-186, or 202-206, a second synthetic RNA oligonucleotide having the nucleic acid sequence SEQ ID NOs:26-30, 46-50, 77-81, 97-101, 132-136, 152-156, 187-191, or 207-211, a third synthetic RNA oligonucleotide having the nucleic acid sequence SEQ ID NOs:31-35, 51-57, 82-86, 102-106, 137-141, 157-161, 192-196, or 212-216, and a fourth synthetic RNA oligonucleotide having the nucleic acid sequence SEQ ID NOs:36-40, 56-60, 87-91, 107-111, 142-146, 162-166, 197-201, or 217-221.

13. A method for stabilizing an siRNA for therapeutic delivery, the method comprising
(a) producing a plurality of synthetic single stranded RNA oligonucleotides complementary to the siRNA sequence, wherein each of the the RNA oligonucleotides comprises 3, 4, 5, 6, 7, 8, 9, 10, 11, 12, 13, 14, 15, 16, 17, 18, 19, 20, or more nucleoside analogs configured to prevent RNase degradation, and wherein each of the RNA oligonucleotides comprises a cholesterol moiety conjugated to its 3′ end, and
(b) contacting the siRNA with the RNA oligonucleotides under conditions suitable to allow hybridization of the siRNA to the RNA oligonucleotides to produce a double-stranded RNA molecules that aggregate together with the cholesterol molecules at the center.

14. The method of claim 13, wherein the nucleoside analogs comprise floxuridine (5-fluorodeoxyuridine, UB5F) nucleotides, gemcitabine (2′, 2′-difluoro-2′-deoxycytidine, dFdC, CR2FF) nucleotides, or a combination thereof.
